(19) United States
(12) Patent Application Publication
(10) Pub. No.: US 2005/0245434 A1
(43) Pub. Date: Nov. 3, 2005

(54) Oxygenated dibenzo-alpha-pyrone chromoproteins

(76) Inventor: Shibnath Ghosal, West Bengal State (IN)

Correspondence Address: Diane Dunn Mckay, Esq.;Mathews, Collins, Shepherd & McKay, P.A., Suite 306, 100 Thanet Circle, Princeton, NJ, 08540, US

(21) Appl. No.: 10/799,104

(22) Filed: Apr. 30, 2004

(51) Int. Cl. A61K 38/17; A61K 38/16; A61K 7/11

(52) U.S. Cl. 514/7; 424/70.14

(57) ABSTRACT

A composition of oxygenated dibenzo-alpha-pyrone chromoproteins (DCP) and their isolation from shilajit, fossils of ammonites, corals and other invertebrates. More particularly, to the description of DCP-composition comprising oxygenated dibenzo-alpha-pyrone or its conjugates, phosphocreatine, proteins, fatty acyl esters of glycerol and other small ligands, e.g., carotenoids, sterols and aromatic acids, as core structural fragments, and their biological functions. Pharmaceutical, nutritional, skin care and personal care formulations are also described. These findings establish DCPs as the major bioactives of shilajit.

### BACKGROUND OF THE INVENTION

1. Field of the Invention

This invention relates to the composition of oxygenated dibenzo-alpha-pyrone chromoproteins (DCP) and their isolation from shilajit, fossils of ammonites, corals and other invertebrates. More particularly, the invention relates to the description of DCP-composition comprising oxygenated dibenzo-alpha-pyrone or its conjugates, phosphocreatine, proteins, fatty acyl esters of glycerol and other small ligands, e.g., carotenoids, sterols and aromatic acids, as core structural fragments, and their biological functions. Pharmaceutical, nutritional, veterinary, skin care and personal care formulations are also described. These findings establish DCPs as the major bioactives of shilajit.

2. Description of the Related Art

There are probably thousands of carotenoproteins to be found in nature. However, even today structures of a very few such compounds has been fully characterized by applying the techniques of protein chemistry. Partial analysis has shown that among these compounds there are many lipoproteins in which the carotenoid moieties appear to be associated also with the lipid component. However, a stoichiometric relationship between carotenoid and protein has not always been found.

This application is related to U.S. Pat. Nos. 6,440,436 B1 and 6,558,712 B1 by the same inventor, which are each incorporated by reference herein.

In many pigmented proteins found in marine invertebrates, —living and fossilized, carotenoids and other coloured compounds (e.g., pyrroloids, biliverdin and indigoids, —indigotin and indirubin) are found to show interaction with the protein part as well as association with the lipid prosthetic group of the complex assembly. But never before has the presence of Oxygenated Dibenzo-alpha-pyrone (DBPs), wherein there is an oxygen linker attached at the 3 and/or 8-position of the DBP, in either free form or in association with chromoproteins, in living or fossilized marine invertebrates, been reported. The present invention describes one such class of pigmented proteins, named dibenzo-alpha-pyronechromoproteins (abbreviated as DCPs), isolated in large abundance, from shilajit, fossils of ammonites, corals and other marine invertebrates.

### SUMMARY OF THE INVENTION

The present invention relates to compositions of DCPs, isolation, and their use in treating various adaptogenic conditions, such as chronic stress.

In one embodiment, the invention provides a composition of dibenzo-alpha-pyrone-chromoproteins (DCPs) which include dibenzo-alpha-pyrone or their derivatives; Phosphocreatine; Chromo-peptides of molecular weights of ≦2 KD; and Lipids having fatty acyl esters of glycerol.

Another embodiment of the invention includes dibenzo-alpha-pyrones of formula (I) 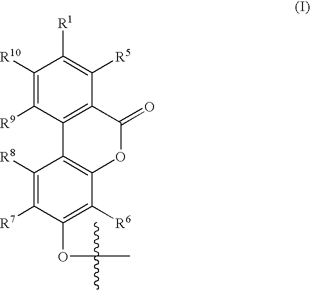
wherein:
R¹ is selected from the group consisting of H, OH, O-acyl, and O-amino-acyl; and R⁵, R⁶, R⁷, R⁸, R⁹, and R¹⁰ are independently selected from the group consisting of H, OH, O-acyl, O-amino-acyl, and fatty acyl groups.

Another embodiment of the invention includes a composition wherein phosphocreatine is attached to the 3- or 8-position of said dibenzo-alpha-pyrones via an ester linkage. Also, the chromo-peptides include one or more amino acids; carotenoids; and indigoids. The chromo-proteins have a molecular weight of about 2 to about 20 KD.

Another embodiment of the invention provides a skin care, hair care, pharmaceutical, veterinary or nutritional formulation comprising a DCP composition present in an amount of about 0.05% to about 50% by weight. Also, the skin care or protection formulation can be in the form of a lotion, cream, gel or spray, wherein the DCP composition is present in an amount of about 0.05% to about 5% by weight.

Another embodiment of the invention provides a pharmaceutical formulation comprising a DCP composition wherein the pharmaceutical formulation is in the form of a tablet, syrup, elixir or capsule.

Another embodiment of the invention provides a nutritional formulation comprising a DCP composition wherein the nutritional formulation contains about 0.5% to about 30% of the DCP composition by weight.

Another embodiment of the invention provides a veterinary formulation comprising a DCP composition wherein the veterinary formulation contains about 0.5% to about 30% of the DCP composition by weight.

Another embodiment of the invention provides a process for isolating DCP compositions from shilajit compositions comprising about 0.5% to about 10% w/w dibenzo-alpha-pyronechromoproteins, the process includes the steps of 1)extracting shilajit successively with hot ethyl acetate and methanol to remove the soluble low and medium molecular weight organic compounds by filtration; 2) triturating the ethyl acetate and methanol insoluble material with hot water and then citrate buffer of pH 5.0; 3) filtering the combined extract-mixture to remove insoluble substances comprising polymeric humic materials, minerals and metal ion salts; 4) gradually saturating the combined aqueous filtrate with increasing concentrations of ammonium sulphate to obtain purple-brown precipitate of mixture of DCPs, or concentrating the combined aqueous solution and adding acetone to precipitate DCPs as brownish-red or off-white precipitate and filtering the DCPs and evaporating the filtrate to obtain an additional lot of mixture of DCPs of lesser complexities; and 5) fractionating the purple-brown solid residues, obtained from ammonium sulphate saturation by Sephadex gel-filtration and electrophoresis to isolate DCP compositions from shilajit.

Another embodiment of the invention provides similar processes for extracting and isolating DCPs from fossils of ammonites, fossils of corals, and from other living and nonliving invertebrates.

Another embodiment provides a method for treating chronic stress disorders, including administering to a patient in need thereof a therapeutically effective amount of a DCP composition and a method for increasing cognition learning which includes administering a DCP composition.

### BRIEF DESCRIPTION OF THE DRAWINGS

FIG. 1A and 1B show the general structure of DCPs and the conjugate assembly of DCPs.

FIG. 2 shows changes in different DCP levels with time in red blood cells of DCP-fed albino rats.

FIG. 3 shows HPLC chromatograms of Shilajit DCPs from ammonium sulphate precipitations.

FIG. 4 shows the relationship between 3, 8-dihydroxy dibenzo-alpha-pyrones and protein fractions.

### DETAILED DESCRIPTION OF THE INVENTION

DCPs, comprising organo-mineral constituents exhibit orange, purple and yellow colors contributed by oxygenated carotenoids known as xanthophylls and indigoids derived from systemic oxidation of tryptophan moieties. The DCPs of shilajit exhibit absorption maxima in the UV and visible regions at λ ˜225, ˜275, ˜320, ˜392, ˜470, ˜492, 500-535, 620-660 nm. An aqueous solution of the DCPs, spread on silica gel having 230-400 mesh, when heated by micro-wave resulted in partial dissociation of carotenoids. The identities of the colored compounds were established by HPLC using authentic markers. The apoprotein part, obtained from this reaction, however, still retained much of the coloring moieties. On gel filtration of the partially degraded protein, and subsequent analysis (e.g., chemical, chromatographic and spectroscopic), of the isolated compounds revealed the presence of a large prosthetic group, particularly rich in DBPs and equivalents.

Selective lipase degradation of the products, liberated DBPs, phospholipids (containing C₁₄-C₂₄ fatty acids, both saturated and unsaturated), and partially cleaved the proteins into chromo-lipoproteins and chromo-apoproteins. Even harsh acidic hydrolysis could not completely detach the nitrogenous constituents from the DBP-nucleus. Thus, the conjugated proteins containing both less polar and more polar fractions still retained some of the amino acids/ small peptides, xanthophylls and indigoids, as determined by HPLC of the degraded products, in the lipase degradation products and some amino acid/small peptide in the conjugate DBPs even after classical acidic hydrolysis.

On saponification, DCPs produced free DBPs and small conjugated DBP metabolites, fatty acids and amino acids. The facile removal of the acylated compounds by saponification suggested that some aminoacyl and fatty acyl moieties are attached to the phenolic hydroxyl group(s) of DBPs. Additionally, the occurrence of small O-acyl conjugates of amino acids in 3-OH-DBP from 3-O-acyl glycinoyl and 3-O-acyl arginoyl DBPs, and also creatine in DCPs support the DBP-prosthetic group structure of the DCPs shown in Formula 1. 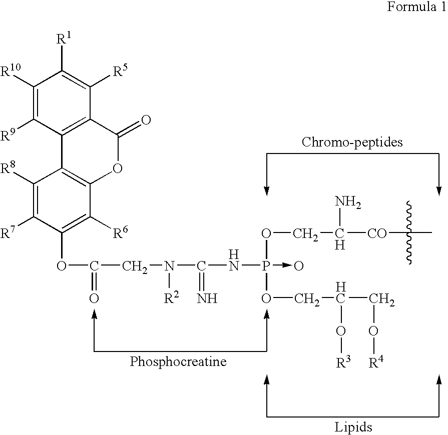
wherein:
R¹=H, OH, O-acyl, O-amino acyl, or di- or tri-peptides of these aminoacids;
R²=H or CH₃;
R³=H or C₁₄-C₂₄ saturated or unsaturated fatty acid; degree of unsaturation ranging from one to six;
R⁴=H or C₁₄-C₂₄ saturated or unsaturated fatty acid; degree of unsaturation ranging from one to six; and
R⁵, R⁶, R⁷, R⁸, R⁹, and R¹⁰ are independently selected from the group consisting of H, OH, O-acyl, O-amino-acyl, and fatty acyl groups.

The chromo-proteins have a weight of 2-20 kilodaltons (KD), and include but are not limited to amino acids, di- and tri-peptides of these aminoacids, carotenoids and indigoids.

Acyclic and cyclic carotenoids or xanthophylls and indigoids, such as lutein, astaxanthin, and beta-carotene are pigments.

Fatty acids may be branched or unbranched and contain carbon atoms between 12 and 20, and may be either saturated or unsaturated. The degree of unsaturation is between one and six.

Degree of unsaturation is the number of double bonds present.

Acyl is —COR where R may be branched or unbranched and contain carbon atoms between 16 and 18, and may be either saturated or unsaturated.

Amino acids include but are not limited to alanine, arginine, creatinine, glycine, hydroxyproline, methionine, proline, serine, threonine, and tryptophan.

A dipeptide results when an amide bond is formed between the —NH₂ of one amino acid and the —COOH of a second amino acid; a tripeptide results from linkage of three amino acids via two amide bonds, and so on. Any number of amino acids can link together to form large chains.

The numbering pattern of the dibenzo-alpha-pyrone is as follows:

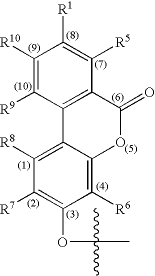

The presence of creatine in DCPs was established by both in vivo and in vitro determinations.

The chromo-moieties in DCPs were found to be associated with both the apolar lipid as well as the polar protein fractions. Lipase degradation followed by characterization of the degraded parts and HPLC analysis showed that the chromo-compounds were attached to the two different fractions albeit in different state of binding. The protein part on further acid hydrolysis produced methionine, arginine, glycine, alanine, serine, threonine, proline and hydroxyproline as the identifiable amino acids.

DCPs contain proteins of molecular weight with a range between 2 to about 20 KD. Separation of DCPs into three bands by polyacrylamide gel electrophoresis (PAGE) revealed that conjugated proteins of molecular weight between about 15 to about 20 KD are present in higher amount than about 2 to about 12 KD. But conjugated protein of molecular weight range about 12 to about 15 KD is present in lowest amount. During elucidating the structures of DCPs, the following striking differences were discerned between the DCPs isolated from shilajit and those from shilajit-precursor-invertebrates:

1. DCPs, in which the apoprotein is colorless, and the colored compounds containing long prosthetic groups (e.g., DBPs and lipids), can be dissociated by simple treatment of aqueous solution of DCPs, either with acetone or ethyl alcohol. The colorless apoproteins exhibit simple HPLC patterns and on acid hydrolysis produced, apart from DBPs and conjugates, the amino acids described above. These DCPs, isolated from fossils of Ammonites, are readily split into the colorless apoproteins and coloring matter, which are soluble in the extracted organic solvents.

2. The other class constitutes DCPs in which the coloring matter comprising carotenoids and indigoids are ordinarily undissociable from the apoprotein. This class of DCPs was isolated from shilajit and from some rare species of fossils of Ammonites (e.g., Perisphinctes with red protoconch)

Proteins of some invertebrates spread at the air/water interface with extreme reluctance. The apoproteins, when dissociated from the prosthetic groups (e.g., containing the coloring matter such as carotenoids), spread smoothly during electrophoresis. The carotenoids in such chromo-proteins seem to act as a ‘lock’ on the tertiary or quaternary structure of the proteins against denaturation. The colorless apoproteins, formed from dissociation of chromoproteins, by contrast undergo immediate coagulation and partial denaturation.

In shilajit-DCPs the association of the chromo-molecules and the apoproteins are not, ordinarily, dissociable. A specific, tenacious, combination of the two moieties is conceivable. Consistent with this postulate, the chromo-compounds in shilajit-DCPs were found to be associated with both the lipid and apoprotein fractions. Selective degradation of DCPs with lipase, followed by HPLC established this point. The stable quaternary structure of the shilajit-DCPs was further suggested by the following experiment. When subjected to electrophoresis in starch-urea gels, two chromoproteins, DCP-I, which is orange-pink in color (Mw≦5 KD) and DCP-II, which is yellowish-brown in color (containing appreciably larger abundance of DBPs than are present in DCP-I; Mw≦14 KD), were separated. These properties suggest that some coloring (pigment) molecules are covalently linked with some parts of the apoproteins and lipo-protein components. A close association between the amino acid moieties, capable of interaction with the carotenoids and indigoids would provide the strength of the association, which in fact is reflected in the profound bathochromic shift (˜λ500 nm to λ660 nm) and hyperchromic effect in the visible spectrum of DCP colored chromophores.

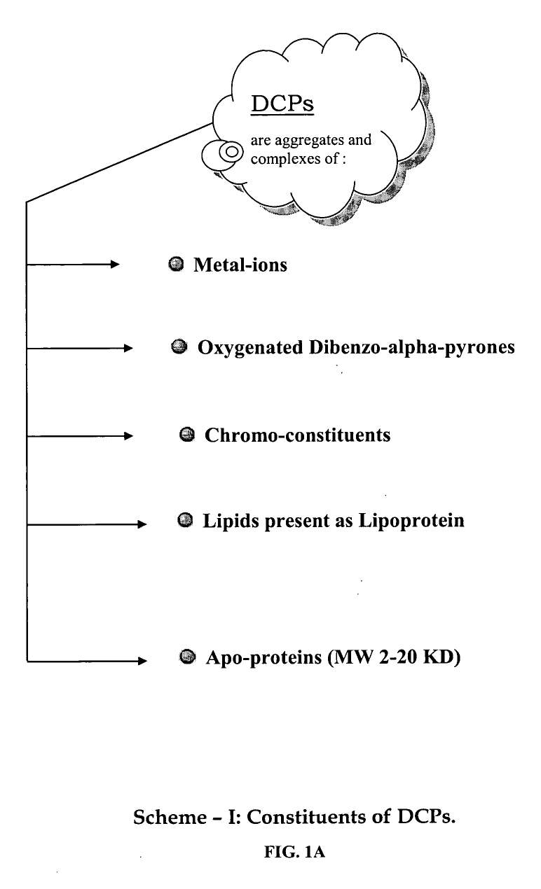

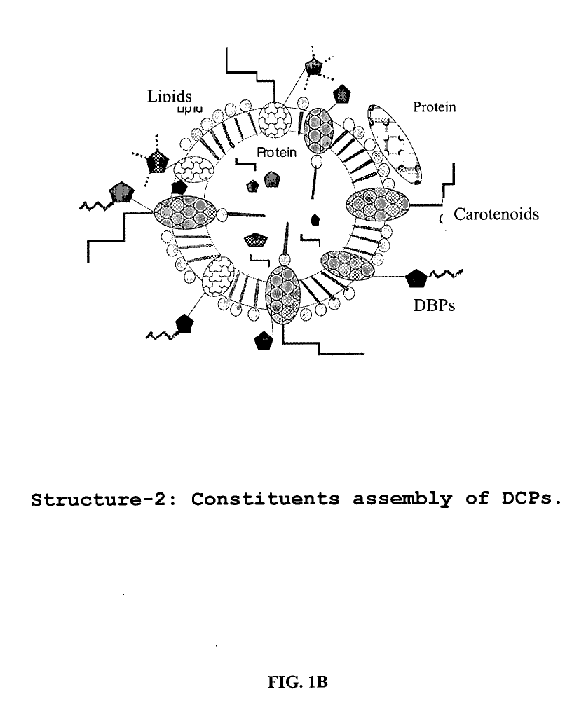

Based on the above, the general structure of DCPs (FIG. 1A) and the conjugate assembly of DCPs (FIG. 1B) were assigned.

The protein content of DCPs, estimated by Lowry's method, was 57.13%; whereas, by the Bradford method it was 59.3%. The higher percentage of protein, estimated by the latter method, was presumably due to its higher sensitivity to the appreciable content of arginine in the DCPs.

Portions of the lipid moieties present in the DCPs (FIGS. 1 and 1A) are covalently linked with the prosthetic group(s). This was suggested by the following study. Exhaustive extractions of DCPs by Bligh and Dyer solvent system, suitable for extraction of lipids, did not yield any free fatty acid but gave a small amount of acylated DCPs. The major insoluble residue on reaction with lipase produced C₁₄ to C₂₄ fatty acids in which C_{16:0}, C_{18:0} and C_{18:1} were the main components as depicted in Table 1. Thus, lipoproteins seem to constitute an integral part of the DCPs.

TABLE 1
Fatty acids composition of four ammonium sulphate
precipitated DCPs after Lipase cut.

| Ammonium | | | | | |
|---|---|---|---|---|---|
| sulphate | Arachidonic | EPA + | 16:0 + | C-14 to C- | C-20 to C- |
| precipitation | acid^{c} | DHA^{c} | 18:0^{c} | 18^{a} | 24^{b} |
| 25% | 14.94% | 4.44% | 0.94% | 17.95% | 82.05% |
| 50% | 20.95% | 10.61% | 0.92% | 18.17% | 81.83% |
| 75% | 0.60% | 9.22% | 27.28% | 48.05% | 51.95% |
| 100% | 0.14% | 4.26% | 1.28% | 43.30% | 56.70% |

^{a+b}= 100% of total fatty acids.
^{c}= Expressed as % of total fatty acids present in each sample.

Many of the shilajit-bearing mountains have been found to be rich storehouses of marine invertebrate fossils, such as of the phyla of Arthropoda, Brachiopoda and Mollusca, of the Phanerozoic era. This co-occurrence of shilajit and the invertebrate fossils, as depicted in Table 2, is a consistent phenomenon.

TABLE 2
Marine invertebrate (fossils and living) analyzed for DBPs and DCPs.

| | | | |
|---|---|---|---|
| | | Age of | |
| | | specimen | |
| | Phylum/Class: | (period) | |
| | Genus, species | Reference/Type | |
| Sr. No | (Order/Family) | number^{a,b} | Place of Occurrence |

Fossils
Arthropoda/Trilobita:

| | | |
|---|---|---|
| I | Ptychoparia spitiensis | Cambrian GSI- |
| | | 9791^{a} |
| II | Asaphus sp. | Ordovician |

Brachiopoda/Articulata:

| | | | |
|---|---|---|---|
| III | Kutchithyris acutiplicata | Jurassic | Kutch, Gujarat |
| | | GSI-6596^{a} | |
| IV | Consinanthris sp. | Cretaceous | Trichy, Tamil Nadu |
| | (Terebratellacea) | | |

Mollusca/Cephalopoda:

| | | | |
|---|---|---|---|
| V | Nautilus angustus | Cretaceous | Ariyaloor, TN |
| | (Ammonoidea) | GSI-97425^{a} | |
| VI | Perisphinctes aberrance | Jurassic | Kutch, GJ |
| | (Ammonoidea) | GSI-2043^{a} | |
| VII | Kamptokephalites | Jurassic | Kutch, GJ |
| | dimerus | JUM-1314^{b} | |
| | (Ammonoidea), | | |
| | female sp. | | |
| VIII | K. dimerus, male sp. | Jurassic | Kutch, GJ |
| | | JUM-1315^{b} | |
| IX | Idiocyclocerus | Jurassic | Kutch, GJ |
| | perisphinctoides | JUM-332^{b} | |
| | (Ammonoidea), | | |
| | female sp. | | |
| X | I. perisphinctoides, | Jurassic | Kutch, GJ |
| | male sp. | JUM-323^{b} | |
| XI | Paryphocerus sp. | Jurassic | Muktinath, Nepal |
| | (Ammonoidea) | | |

Foraminifera (Protozoa):

| | | | |
|---|---|---|---|
| XII | Alveolina sp. | Cretaceous | Kutch, Gj |
| XIII | Discocyclina sp. | Paleocene, | Javana, Trichi |
| | | Oligocene | |
| XIV | Nummulites sp. | Early Miocene | Kutch, Gj, yanthia |
| | | | Hill, India |
| XV | Nacutus sp. | — | Kutch, Gj |

Cnidaria/Anthozoa (coral)

| | | | |
|---|---|---|---|
| XVI | Diploria | — | Bay of Bengal |

Cnidaria/Hydrozoa (coral)

| | | | |
|---|---|---|---|
| XVII | Stylaster | — | Bay of Bengal |

Marine invertebrates (living)

| | | | | |
|---|---|---|---|---|
| | Phylum/Class: | Age of specimen | | |
| Sr. | Genus, species | (period), Reference/ | Place of | Parts |
| No | (Order/Family) | Type number^{a,b} | Occurrence | examined |

Living invertebrates - Mollusca/Gastropoda

| | | | | |
|---|---|---|---|---|
| XIX | Telescopium | — | Coastal | Body flesh |
| | telescopium | | region of | |
| | | | Bay of | |
| | | | Bengal | |
| XX | Cerethedia | — | Coastal | Body flesh |
| | cingulata | | region of | |
| | | | Bay of | |
| | | | Bengal | |

Mollusca/Cephalopoda

| | | | | |
|---|---|---|---|---|
| XXI | Loligo sp. | — | Coastal | Body flesh |
| | | | region of | |
| | | | Bay of | |
| | | | Bengal | |

Arthropoda/Crustacea

| | | | | |
|---|---|---|---|---|
| XXII | Osipoda | — | Coastal | Body flesh |
| | macrocera | | region of | |
| | (Red rab) | | Bay of | |
| | | | Bengal | |
| XXIII | Copepoda | — | Coastal | Body flesh |
| | | | region of | |
| | | | Bay of | |
| | | | Bengal | |

^{a}Geological Survey of India, Calcutta
^{b}Geological Sciences Museum, Jadavpur University, Calcutta (through the courtesy of Prof. S. Bardhan)
The remaining samples were obtained from Messrs Hindusthan Minerals, Calcutta.

Also, the organic compounds found in these fossils and in shilajit are very similar as shown in Tables 3-6.

TABLE 3
HPTLC data of compounds found common in marine
invertebrates and shilajit

| | Developing | | Reflectance | Mode of |
|---|---|---|---|---|
| Compound | Solvent | R_{F} | max./nm | detection |
| 3-Hydroxy-DBP | A | 0.51 | 222, 230, 278, | D-Q/M-F |
| | | | 300, 330 | |
| Monoacyl-3,8- | A | 0.35 | 218, 252, 304, | D-Q/M-F |
| dihydroxy-DBP^{a} | | | 330, 355 | |
| 3,8-Dihydroxy-DBP | A | 0.22 | 215, 236, 272, | D-Q/M-F |
| | | | 294, 352 | |
| Dimeric-DBP | B | 0.25 | 215, 280, | D-Q |
| | | | 332, 348 | |
| Glucitol | B | 0.20 | — | T, BMP |
| Ribitol | B | 0.18 | — | T, BMP |
| Allantoin | C | 0.42 | 228, 262 | D-Q |
| Uric acid | C | 0.33 | 222, 288 | D-Q |
| Proline | C | 0.25 | — | T, Nin. |
| Hydroxyproline | C | 0.20 | — | T, Nin. |
| Glycine | C | 0.16 | — | T, Nin. |

^{a}the acyl moiety was constituted of C₁₆-C₂₀ fatty acids
Q quenching mode
D deuterium lamp, wave length 260 nm
M mercury lamp, wave length 360 nm;
F, fluorescence mode
T tungsten lamp, wave length 520 nm;
BMP, benzidine-metaperiodate staining reagent for polyols, sugars;
Nin, ninhydrin reagent for detection of amino acids

TABLE 4
HPLC data of compounds found common in marine
invertebrates and shilajit

| | Retention time | |
|---|---|---|
| Compound | t_{R} in min | PDA λₘₐₓ nm |
| 3,8-Dihydroxy-DBP dimer | 5.55 | 238, 295, 318, 337, 375 |
| 2,4,6 | 6.22 | 222, 268, 318, 342 |
| Trihydroxyacetophenone | | |
| 2,4- | 6.41 | 220, 230sh, 263, 275, 325 |
| Dihydroxyacetophenome | | |
| 3,5- | 6.50 | 218, 263, 315 |
| Dihydroxyacetophenone | | |
| Benzoic acid | 8.197 | 228, 272 |
| 3,8-Dihydroxy-DBP | 10.08 | 238, 271, 280, 300, 350 |
| 3,8-DBP-quinone | 11.33 | 220, 230, 290, 345, 390 |
| Monoacyl-3,8-dihydroxy- | 25.68 | 243, 290, 304, 342 |
| DBPs^{a} | | |
| 3-Hydroxy-DBP | 31.06 | 233, 271, 295, 304, 330 |

^{a}C₁₆-C₂₀ fatty acids were detected after hydrolysis followed by GC of their methyl esters using markers

TABLE 5
GC-MS data of compounds found common in marine invertebrates and shilajit

| | | Retention time, | |
|---|---|---|---|
| Compound | Mol. formula | t_{R} in min | MS: m/z |
| Dotriacontanol | C₃₂H₆₀O | 11.032 | 466 (M⁺) |
| o- | C₉H₁₀0₂ | 12.110 | 150 (M⁺), 135, 107, 92 |
| Methoxyacetophenone | | | |
| EPA as methyl ester | C₂₁H₃₂O₂ | 19.033 | no detectable M⁺, |
| | | | fragment-ions: 287, |
| | | | 284, 279, 274, 262, |
| | | | 201, 187, 105, 91 |
| Dotriacontane | C₃₂H₆₆ | 19.048 | 450 (M⁺) |
| Oleoyl alcohol | C₁₈H₃₆O | 22.751 | 268 (M⁺) |
| Hentetracontanol | C₄₁H₈₄O | 22.899 | 592 (M⁺) |
| DHA as methyl ester | C₂₃H₃₄O₂ | 23.150 | no detectable M⁺, |
| | | | fragment-ions: 268, |
| | | | 262, 254, 247, 223, |
| | | | 219, 105, 91 |
| Methyl-4- | C₁₉H₃₈O | 23.416 | 380 (M⁺) |
| hydroxyoctadecanoate | | | |
| Tetratetracontane | C₄₄H₉₀ | 23.567 | 618 (M⁺) |
| Squalene | C₃₀H₅₀ | 26.56/26.716 | 410 (M⁺), 395, 367, |
| | | | 341, 299, 175, 149, |
| | | | 123, 105, 95, 69 |
| Dinosterane | C₃₀H₅₄ | 27.821 | 414 (M⁺), 301, 300, |
| | | | 273, 272, 177, 93 |
| 24-Ethylcholestane | C₂₉H₅₂ | 30.042 | 400 (M⁺), 287, 286, |
| | | | 269, 268, 229, 117, 85 |
| Benzamide | — | 5.719 | 193 (M⁺), 178, 105, |
| | | | 77, 73 |
| Phenylacetic acid | — | 6.06 | 208 (M⁺), 193, 118, |
| | | | 91, 77 |
| m-Hydroxybenzoic | — | 6.384 | 282 |
| acid (as Di-TMS) | | | (M⁺), 267, 223, 193, 147, 73 |
| N-Methyl hippuric acid | — | 6.70 | 265 (M⁺), 250, 206, |
| | | | 190, 177, 105, 73, 51 |
| 2- | | 6.749 | 208 (M⁺), 193, 180, |
| Hydroxyacetophenone | | | 151, 105, 73 |
| 2,4- | — | 7.283 | 296 (M⁺), 281, 252, |
| Dihydroxyacetophenone | | | 239, 179, 73 |
| (as mono- | | | |
| TMS) | | | |
| Ribitol (as penta- | — | 7.480 | 512 (M⁺), 413 (base |
| TMS) | | | peak) |
| p-Hydroxy-N- | — | 7.666 | 223 (M⁺), 208, 178, |
| methyl benzamide | | | 177, 151, 150, |
| (as mono-TMS) | | | 119, 73 |
| Glucitol (as hexa-TMS) | — | 7.698 | 614 (M⁺), 485 (base |
| | | | peak), 319, 205 |
| m-Hydroxyphenyl | — | 7.984 | 310 (M⁺), 295, 251, |
| propionic acid (as di- | | | 194, 117, 73 |
| TMS) | | | |
| 3-Hepten-4- | — | 9.551 | 390 (M⁺), 375 (base |
| hydroxydioic acid (as | | | peak), 259, 244, |
| di-TMS) | | | 117, 73 |
| m-Cresol | — | 10.59 | 180 (M+), 165, 79, 51 |
| Uric acid (as tetra- | — | 14.203 | 456 (M+), 441 (base |
| TMS) | | | peak) 426, 383, 367, |
| | | | 147, 77, 73 |
| 3-Hydroxy-DBP | — | 18.702/19.851^{a} | 284 (M+), 269 (base |
| | | | peak), 241, 213, |
| | | | 183, 156, 94, 75 |
| 3,8-Dihydroxy-DBP | — | 23.910/25.165^{a} | 372 (M+), 357, 327, 73 |
| p-Hydroxy-bis- | — | 32.533 | 344 (M+), 329, 179, |
| diphenyl methane (as | | | 157, 135 |
| di-TMS) | | | |
| Cholesterol | — | 36.283 | 458 (M+), 443 (base |
| | | | peak), 368, 329, 247, |
| | | | 213, 129, 73 |

^{a}GC-MS in two different conditions

TABLE 6
Relative abundance of different groups of compounds^{a} found
in marine invertebrate fossils and in shilajit

| | Relative abundance % | | |
|---|---|---|---|
| Compound type | Foraminifera^{b} | Mollusca^{c} | Shilajit^{d} |
| Hydrocarbons | 5.46 | 2.08 | 4.03 |
| Fatty acids | 15.10 | 14.77 | 11.56 |
| Wax esters | 1.33 | 2.05 | 3.88 |
| Alkyl glycerols | 0.88 | 0.76 | 0.57 |
| Alkylacylglycerols | 1.04 | 1.11 | 2.58 |
| Triacylglycerols | 2.11 | 3.54 | 5.03 |
| Aromatic/phenolic | 4.45 | 9.21 | 12.10 |
| acids | | | |
| Hydroxyacetophenones | 0.24 | 2.31 | 2.39 |
| N,S-Heterocyclics | 0.18 | 2.74 | 1.01 |
| Oxygenated DBPs | 14.55 | 8.31 | 3.03 |
| DBP-Chromoproteins | 2.01 | 21.10 | 32.33 |
| (DCPs) | | | |
| Partially characterized | 7.22 | 11.60 | 8.64 |
| compds. | | | |
| Humic substances | 45.43 | 20.42 | 12.85 |
| (including polymeric | | | |
| compds)^{e} | | | |

^{a}By GC-MS analysis of corresponding methyl esters and TMS derivatives and other chromatographic and spectroscopic analyses
^{b}Mean of rel. abundance of compounds isolated from Nummulites, Alveolina, and Discocyclina fossils
^{c}Mean of rel. abundance of compounds isolated from fossils of Mollusca
^{d}Collected from the Kumaon region of the Himalaya
^{e}Estimated by HPTLC

These findings suggest that marine invertebrates contribute to the formation of shilajit.

The marine invertebrates (Table 2) were investigated, followed by the isolation and characterization of DCPs in shilajit. Very similar DBP-carotenoproteins and other low Mw organic and coloring constituents (e.g., indigoids) were found in the marine invertebrate samples (Table 3-6).

The IR spectra of the mixture of DCPs isolated from shilajit and the Ammonites (Table 2) were very similar. Also, the HPLC retention times of the major peaks and their PDA spectra. The DCP-fractions on exhaustive organic solvent extractions followed by the usual work-up yielded astaxanthin, astaxanthin fatty acyl derivatives and canaxanthin. 3,8-Dihydroxy dibenzo-alpha-pyrone and the amino acids isolated from shilajit-DCPs, were also isolated from the Ammonite fossils (Table 2) from their acid hydrolysates.

The colored constituents of the DBP-chromoproteins from the Ammonites included mono-N-benzoyl indigotin, indirubin and isatin, presumably derived from the metabolism of the tryptophan moiety present in the DCPs. The browning of the proteins from the glycation of proteins, due to oxidative stress, was also discerned in the DCPs of both shilajit and the Ammonites fossils.

Preservation of color patterns on invertebrate fossils is a rare phenomenon but has been recorded throughout the Phanerozoic. The colored molecules comprising carotenoids, indigoids, and glycation of protein products, by the Maillard reaction, may form stable complexes by coordination with metal ions. Such intra-crystalline biomolecules act as a nucleation site for biomineralization. When limb muscles of dead marine animals decay, the vacated spaces are filled with minerals, such as pyrite (FeS₂, CaSiO₃) before the thin organic cuticles that surround them have time to collapse or decay. The organic material forms a substrate for the nucleation of pyrite (and other minerals), which is ubiquitous in marine sediments. Precipitation is ensued as a result of diffusion of Fe and S into the cell. Pyrite does not replace the tissue directly but precipitates on surfaces and within spaces. Mutual stabilization of the coloured molecules and proteins in shilajit as well as in the fossils of Ammonites, was augmented by the participation of the DBPs (FIGS. 1 and 1A). This is the first demonstration of the natural occurrence of DBPs in complex association with chromoproteins. Whether this association is a general phenomenon, also in the living human and animal organisms, was also evaluated. Mixture of DCPs (pink colored) isolated from plasma of albino rats when compared with the corresponding fractions of DCPs from shilajit exhibited some striking similarities in respect of HPLC peaks and their PDA spectra. Even greater similarities were observed between semi-purified DCP constituents by gel filtration over Sephadex G-50, obtained from shilajit and from the plasma of a human volunteer. Similar general HPLC patterns were observed with several healthy human subjects.

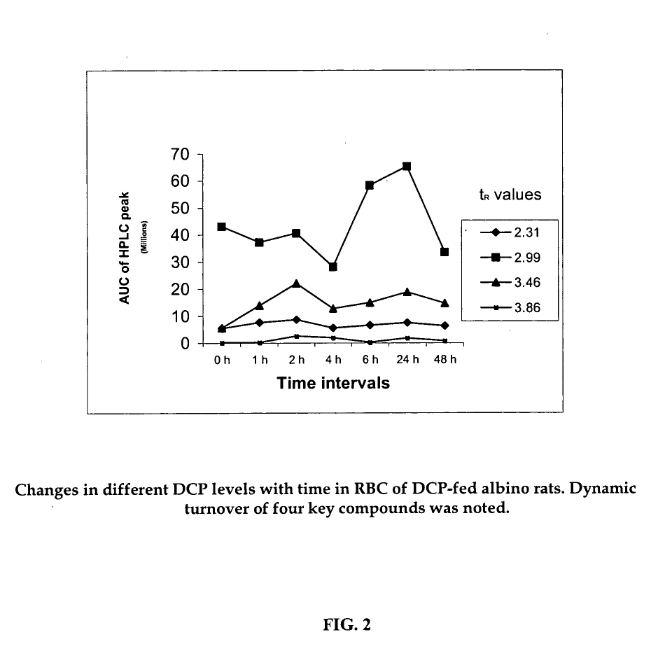

DCPs when administered to experimental animals showed dynamic turnover in respect of some of the key constituents (FIG. 2). Likewise, the DBPs when administered orally (p. o.) to rat readily absorb and utilized them for the synthesis of DCPs and related conjugates. Oxygenated dibenzo-alpha-pyrones (DBPs), on being synthesized in the animal living systems from EPA, are transformed into several DBP-conjugates (HPLC-t_{R}: 2.31, 2.99, 3.46 and 3.86 min). These components were also detected in DCPs, isolated from shilajit. A dynamic turnover of these constituents was observed (FIG. 2) on oral administration of DCPs (200 mg/Kg b.w.) to albino rats, followed by HPLC analysis of the constituents in the corresponding RBC. From this and other observations, it is increasingly apparent that DCPs, which are also the constituents of animal tissues, act in the form of enzymes and hormones in regulating and fulfilling several biological functions.

DCPs may participate in a variety of functions in the producer organisms including protective-colorations which provide protection from radiation, electron transport, and enzyme activity and in their sustenance and development. DCPs, which have transport properties like those of the fulvic acids (FAs) of shilajit, can enter into recipient cells and elicit biological responses much more pronounced than free DBPs. Extensive pharmacological and immunological evaluations of DCPs have now demonstrated them to be 2-5 times more potent than any of the other constituents of shilajit as adaptogen and immunomodulator.

The systemic transformation of 3-hydroxy- and 3,8-dihydroxydibenzo-alpha-pyrone (DBPs) into arginine and glycine phospholipid conjugates, their resultant metabolism, and the systemic assimilation/turnover of DCPs, when fed to rats through oral route, suggest the role of these compounds in energy storage in living systems. Arginine phosphate plays an important role in the storage of energy in invertebrates; the same role is played by creatine produced from a combination of argininephosphate and glycine phosphate in vertebrates. Creatine phosphate and arginine phosphate are reserves of phosphates of high energetic potential and, hence, the name ‘phosphagens’ given to these compounds as shown in Scheme 1.

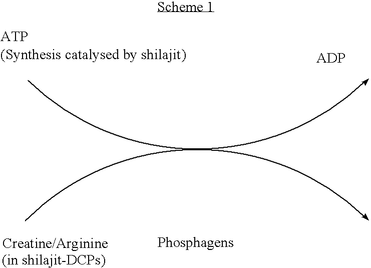

An energetic coupling represents the energy storage reaction when ATP is present in excess and, inversely, the formation of ATP by the reverse reaction when the cells need the ATP. Should we consider the biosynthesis and balance of DBP-phosphagen complexes in living organisms as the indices of their energy status, then in the event of death of these phosphagens, administration (p. o.) of shilajit would replenish them.

The chromoproteins (DCPs), participate in a wide variety of functions in animal biological systems. DCPs have been encountered in the lowest form of animal organisms (foraminifera, in other marine invertebrates, and in haemolymph of termites), in higher animals (rodents, beaver, chimpanzee, sheep), and in man.

DCPs participate in electron transport systemic ATP synthesis by DCPs is conceivable because oral administration of DBP produced creatine and conjugated product(s) and oxido-reductase reactions; catalyze other enzyme activities (e.g., ATPase function as described in Cheesman, 1967); the larger abundance of DCPs in female invertebrate fossils of the Jurassic (e. g., Idiocyclocerus and Kamptokephalites spp.) (Table 2) compared to their male counterparts, found in the present study, suggests their role in the development and protection of the embryos. The superior (qualitative and quantitative) biological functions of the DCPs compared to those of EPA, DHA, and free DBPs formed from EPA/DHA are described in the sequel.

Thus, features of the isolation and use of DCPs are as follows:

1. Stabilization of protein and the colored molecules, carotenoids (e.g., astaxanthin and derivatives) and indigoids (e.g., indigotin and indirubin) against different forms of stress and onslaughts.

2. Protective coloration, —the use of color as a means of concealment from prey-predator functions; utilization of the potently antioxidant pigments from deleterious effects of radiation; e.g., photo-oxidation of lipids, and from oxidative free radicals.

3. Development of the producer organisms. The large number of pigmented proteins which have been found in the ovaries of invertebrates and the higher abundance of these compounds in the female species compared to those of the male counterparts, suggest their function in the species development. Lipoprotein complexes which have been noted in the blood/haemolymph of many invertebrates may be involved in the transport of the carotenoids and other pigment molecules; the linkage to a protein making the fat-soluble pigments water-soluble. Hence the chromo-molecules in DCPs were found associated with both the lipid as well as the protein fractions of the complex molecules.

4. Development of embryos in invertebrates require carotenoproteins.

5. As simulator/surrogates of bio-energetics, e.g., ATP; creatine synthesis.

6. Immuno-modulator.

7. Captivators of oxidative free radicals, Reactive Oxygen Species (ROS), Reactive Nitrogen Species (RNS).

8. Scavengers/chelators of loose metal ions (Fe, Cu, Mn, W).

9. DCPs play a crucial vitalizer role in all organisms since the evolution of life on Earth.

The features of the isolation and use of DCPs provides a skin care, hair care, pharmaceutical, or nutritional formulation comprising a DCP composition present in an amount of about 0.05% to about 50% by weight. Also, the skin care or protection formulation can be in the form of a lotion, cream, gel or spray, wherein the DCP composition is present in an amount of about 0.05% to about 5% by weight.

The features of the invention provide a pharmaceutical formulation comprising a DCP composition wherein the pharmaceutical formulation is in the form of a tablet, syrup, elixir or capsule.

The features of the invention provides a nutritional formulation comprising a DCP composition wherein the nutritional formulation contains about 0.5% to about 30% of the DCP composition by weight.

The features of the invention provides a veterinary formulation comprising a DCP composition wherein the veterinary formulation contains about 0.5% to about 30% of the DCP composition by weight.

The features of the invention provides a process for isolating DCP compositions from shilajit compositions comprising about 0.5% to about 10% w/w dibenzo-alpha-pyronechromoproteins, the process includes the steps of 1)extracting shilajit successively with hot ethyl acetate and methanol to remove the soluble low and medium molecular weight organic compounds by filtration; 2) triturating the ethyl acetate and methanol insoluble material with hot water and then citrate buffer of pH 5.0; 3) filtering the combined extract-mixture to remove insoluble substances comprising polymeric humic materials, minerals and metal ion salts; 4) gradually saturating the combined aqueous filtrate with increasing concentrations of ammonium sulphate to obtain purple-brown precipitate of mixture of DCPs, or concentrating the combined aqueous solution and adding acetone to precipitate DCPs as brownish-red or off-white precipitate and filtering the DCPs and evaporating the filtrate to obtain an additional lot of mixture of DCPs of lesser complexities; and 5) fractionating the purple-brown solid residues, obtained from ammonium sulphate saturation by Sephadex gel-filtration and electrophoresis to isolate DCP compositions from shilajit.

The features of the invention provides similar processes for extracting and isolating DCPs from fossils of ammonites, fossils of corals, and from invertebrates.

The features provide a method for treating chronic stress disorders, including administering to a patient in need thereof a therapeutically effective amount of a DCP composition and a method for increasing cognition learning which includes administering a DCP composition.

The following examples will serve to further typify the nature of the invention.

### EXAMPLE 1

### Extraction and Isolation of DCPs of Shilajit

Shilajit (rock powder) was extracted successively with hot ethyl acetate and methanol to remove free organic compounds which were subsequently analyzed comprehensively (Tables 2-5). The marc (ethyl acetate- and methanol-insoluble material) was triturated with hot water and citrate buffer (pH 5.0) and then filtered. The marc was analysed for inorganic minerals and humic substances. The aqueous solution was differently saturated with ammonium sulfate (25%, 50%, 75% and 100%) when DCPs of different complexities were precipitated as purple-brown solid. The solid residues were subjected to Sephadex gel filtration and electrophoresis for further purification of DCPs. The same general procedure was followed for the isolation of DCPs from the marine samples. In the precipitation of DCPs from aqueous solutions, however, one variation constituted addition of acetone, instead of ammonium sulfate and to isolate DCPs from acetone-insoluble and soluble fractions in the usual way.

### EXAMPLE 2

### Extraction and Isolation of DCPs of Marine Invertebrate Fossils (General Procedure)

In a typical experiment, fossils of Nummulites (foraminifera, GSI type No. 10772) were dried, finely powdered and then extracted with hot ethyl acetate to remove low Mw organic compounds (free oxygenated dibenzo-alpha-pyrones, hydroxyacetophenones, aromatic acids etc., cf. Tables 3-6) as the ethyl acetate-soluble fraction. The marc (insoluble in ethyl acetate) was further extracted with 0.1N HCl. The aqueous acidic extract was evaporated. The residue was dissolved in minimum volume of distilled water. The aqueous solution was divided into two parts. One part was differently saturated with ammonium sulfate and to the other part, acetone was gradually added. Addition of both ammonium sulfate and acetone precipitated mixtures of oxygenated dibenzo-alpha-pyrone chromoproteins (DCPs) as light brown solid. The acetone-soluble fraction, on evaporation also afforded a further crop of DCPs of lesser complexities. These compounds were subsequently subjected to chromatographic (HPLC) and spectroscopic (IR, ¹H-NMR) analyses to establish their general identities with DCPs.

### EXAMPLE 3

### Extraction of Living Marine Invertebrates (General Procedure)

Living marine invertebrates mainly molluscs (Telescopium, Cerethedia etc.) were collected from coastal region of Bay of Bengal and brought to the laboratory as live specimen. Each specimen was sacrificed and body flesh was taken out from shell. Body flesh was then extracted with hot ethylacetate to remove low molecular weight organic compounds and lipids. The marc (EtOAc insoluble portion) was further extracted with Bligh & Dyer solvent system [CHCl₃: MeOH (1:2) as initial solvent; CHCl₃: MeOH: H₂O (1:2:0.8) as intermediate solvent and CHCl₃: MeOH (1:2) as final solvent]. The Bligh & Dyer (D&B) solvent was evaporated under reduced pressure. The B&D extractive was dissolved in minimum volume of distilled water. The aqueous solution was divided into two portions. One portion was gradually saturated with ammonium sulphate and to the other portion, acetone was gradually added. Addition of both ammonium sulphate and acetone precipitated mixtures of DCPs (oxygenated dibenzo-alpha-pyrone chromoproteins) as off white solid. These compounds were analyzed by different chromatographic (HPLC) and spectroscopic (IR, ¹H-NMR, GC-MS) techniques to establish their identities with shilajit DCPs.

### EXAMPLE 4

### Separation and Partial Characterization of DCPs (I and II)

Sodium dodecyl sulfate polyacrylamide gel electrophoresis was carried out by the method of Weber and Osborn (1969) with 10% acrylamide in presence of 0.1% (w/v) SDS. The sample was preheated at 100° C. for 3 minutes in presence of 2-mercaptoethanol and 3% SDS. Tris-glycine buffer containing 0.1% SDS (pH 8.4) was used as running buffer. Bromophenol blue was used as tracking dye. Electrophoresis was performed at a constant current of 120V, 40 mA for 90 min. A pinkish-orange band appeared towards the top (DCP-I) followed by bromophenol blue and then a yellow band (DCP-II). After the run was over, these three bands were cut with a fine blade and homogenized, separately, in 1.5 ml distilled water in a mortar-pestle. Each homogenate was decanted into a micro-centrifuge tube and centrifuged at 7000 rpm for 10 minutes. Each supernatant was divided into two parts and evaporated under vacuum. One part of the sample (ca. 50 μg) was dissolved in HPLC running solvent (water:acetonitrile:orthophosphoric acid=67:32:1) and analysed by HPLC. The other part was subjected to lipase reaction (see EXAMPLE 5).

The DCP-I compound was obtained as a pink colored powder; pH (1% aqueous solution) 8.02; N, 17.8%; metal ions (in ppm) Fe, 186.3; Cu, 8.8; Zn, 23.4.

The DCP-II compound was obtained as a light brown powder, pH (1% aqueous solution) 7.8; N, 16.4%; metal ions (in ppm) Fe, 262.4; Zn, 48.7.

Further purification of the two chromoproteins was carried out by Sephadex ion exchange on DEAE-Sephadex G-50, using phosphate buffer (pH 7.2). Gel electrophoresis (10% SDS, thickness 1.5 cm; constant current 20 mA, tris-glycine buffer, pH 8.3) showed two major bands in each of DCP-I, 2-5 KD and DCP-II, 10-14 KD; with several lighter bands at higher Mw ranges.

Both DCP-I and DCP-II exhibited HPLC and spectroscopic (IR, ¹H-NMR) characteristics typical of DBP-carotenoproteins.

### EXAMPLE 5

### Lipase Reaction of DCPs

The sample (ca.50 μg) was dissolved in 0.5 ml 1M tris-buffer of pH 8.0.100 μl (2.2%) CaCl₂.2H₂O and 250 μl (1%) bile salts were added to each sample. Working solution of lipase (Hog pancreatic lipase, Sigma, 1 mg in 2 ml tris-buffer) was then added to each sample. The mixtures were agitated by magnetic stirrer for three hours at 370° C. After the incubation period, 1 ml ethanol and 1 ml 6N HCl was added to the mixtures to stop the reaction. The hydrolyzed products were extracted by diethyl ether and dried over anhydrous sodium sulfate. The remaining portions were evaporated on water bath in porcelain basin. The residues were dissolved in minimum volume of HPLC solvent (water:acetonitrile:orthophosphoric acid=67:32:1) and 20 μl was injected into HPLC for analysis. Collective ether extractives, after lipase hydrolysis, was also analysed in HPLC in the same solvent system to characterize the nature of lipoidal compounds.

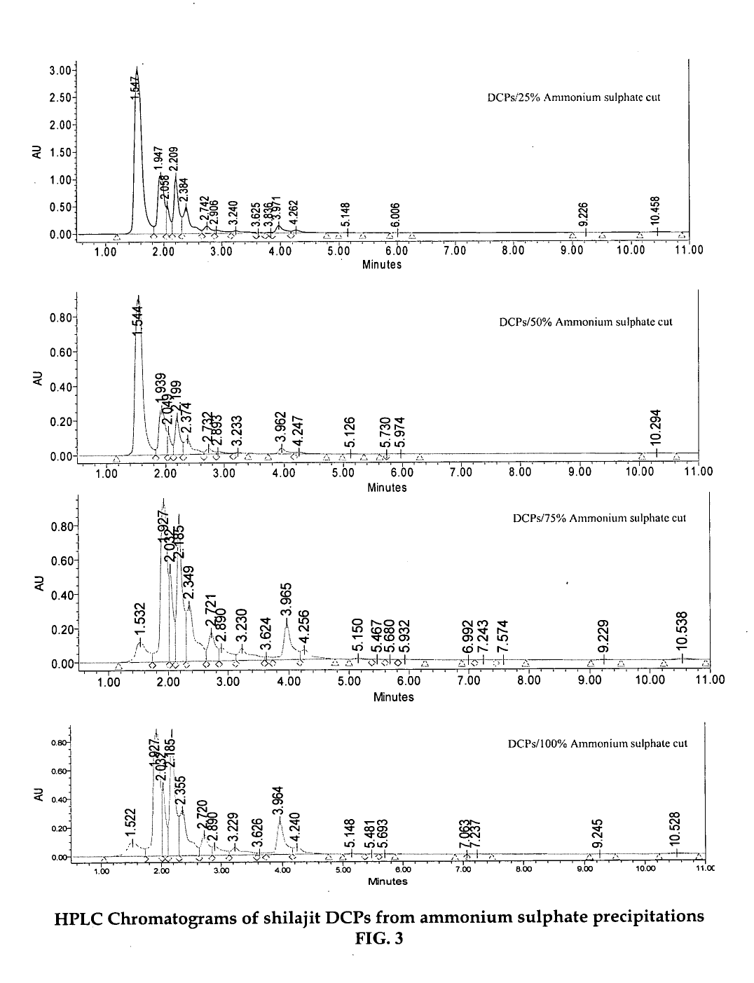

In the HPLC chromatograms of DCPs, precipitated from aqueous solution of shilajit by differently saturating with ammonium sulfate, a large number of peaks appeared in the 75 and 100 percent-saturated fractions (FIG. 3). This observation suggested that shilajit DCPs are replete with relatively low Mw lipoproteins (like chylomicrons/lipocalins). However, t_{R} 1.5 min signal (FIG. 3) suggested that higher Mw proteins, like B-48, might also occur in DCPs. The presence of adherent ligands, particularly DBPs, was also suggested.

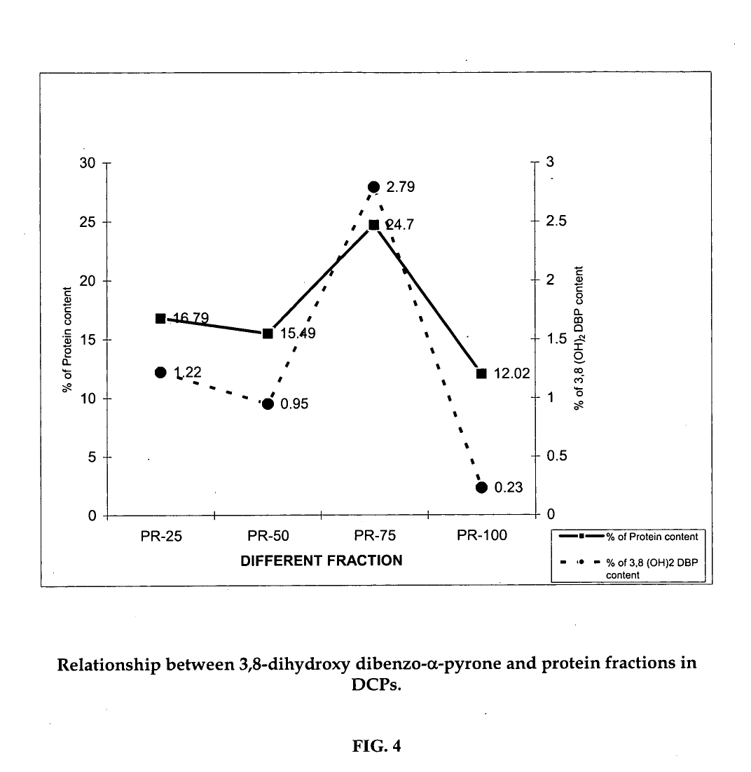

Another observation was the association of DBPs as ligands in DCPs (FIG. 4). In this figure, PR-25, -50, -75 and -100 denote respective ammonium sulfate precipitated protein fractions. Note that in the PR-50 and -75, the abundances of 3, 8-dihydroxydibenzo-alpha-pyrone are very high suggesting that the DBPs are preferentially associated with low/medium MW lipoproteins.

### EXAMPLE 6

### Determination of Amino Acids

The mixture of amino acids produced in the acidic hydrolysates of DCPs was converted into trimethylsilyl derivatives (O-/N-TMS) and then subjected to GC-MS analysis by using corresponding markers, similarly prepared with the standard amino acids.

### EXAMPLE 7

### Determination of Creatine

This method, based on the color reaction developed by creatine in the presence of diacetyl and a-napthol, was described by Barrett (1936). Briefly, to a neutral solution of the test sample, containing not more than 60 μg of creatine, 2 ml of 1% α-napthol in alkali was added followed by 1 ml of diacetyl (1% solution diluted to 1:20 before use). The solution was shaken, and the color was measured after 30 min at 525 mμ.

### EXAMPLE 8

### Determination of Arginine

Arginine, isolated from DCPs by selective degradation (lipase), was decomposed by arginase (5 to 10 units/ml) to ornithine and urea and were assayed calorimetrically (using acid mixture, —1 vol. H₂SO₄; 3 vol. syrupy H₃PO₄; 1 vol. H₂O; urea standard, 50 μg/ml in H₂O; and α-isonitrosopropiophenone, 4 g. in 100 ml of 95% ethyl alcohol).

### EXAMPLE 9

### Comparative Study of the Effects of Shilajit Constituents on Chronic Stress

A comparative study of shilajit bioactive constituents from EPA, DHA, DBPs and DCPs, was carried out to determine their adaptogenic potency against chronic stress (CS) in albino rats. It is now increasingly becoming evident that CS of a mild but unpredictable nature which the animal is unable to cope with (inescapable stress), is clinically more relevant than acute stress even when the latter is severe in nature. It is believed that chronic, unpredictable, and inescapable stress resembles the situation faced by an individual that ultimately results in chronic stress- induced physiological perturbation and disease.

Animals The investigation was carried out on CF strain albino rats, of either sex (140-180 g), housed in colony cages at an ambient temperature of 25±2° C., with a 12 h. light /12 h. dark cycle. Experiments were conducted between 0900 and 1400 hrs.
EPA, Eicosapentaenoic acid
DHA, Docosahexaenoic acid
DBPs, 1:1 mixture of 3-hydroxy- and 3,8-dihydroxydibenzo-alpha-pyrone
DCPs, DBP-chromoproteins

### Induction of Chronic Stress

The procedure of Armario et al (1993) was followed. Briefly, rats were randomly assigned to control or stress groups. Those assigned to the stress groups were subjected to 1 h foot shock, through a grid floor, every day for 14 days. The duration of each shock (2 mA) and the intervals between the shocks were randomly programmed between 3-5 sec. and 10-110 sec., respectively, to make the stress unpredictable. The shock chamber had high walls which made escape from shock impossible.

### Test Compounds and Vehicles

EPA (Aldrich, Milw.), DHA (Sigma), DBPs and DCPs were separately suspended/dissolved in 0.3% carboxymethylcellulose(CMC) in distilled water and administered orally (p.o.) , for 14 days, starting on day 1, 60 min prior to electroshock. Control animals received only the vehicle in either unstressed or the stressed rats for the same period in a volume of 2.5 ml/kg, p.o. Estimations were conducted on day 14, one hour after the last stress procedure and two hours after the last test compound or vehicle was administered.

### Determination of Intensity of Chronic Stress Effects

Gastric ulcerations (Bhattacharya et al., 1987). On day 14, rats were killed by decapitation the stomach was split open along the greater curvature and the numbers of discrete ulcers were noted. The severity of ulcers was scored, after histological confirmation, as 0=no ulcers; 1=changed limited to superficial layers of mucosal with no congestion; 2=half the mucosal thickness shows necrotic changes; and 4=complete destruction of mucosa with hemorrhage. Thereafter, the pooled ulcer score was calculated according to the method of Bhattacharya et al. (1987).

### Adrenocorticoid Activity

Adrenal gland ascorbic acid (Zenker and Bernstein, 1958) and corticosterone concentrations (Selye, 1936), and plasma corticosterone levels (Selye, 1936) were determined to substantiate the validity and intensity of the stress procedure adopted.

### Results and Discussion

Chronic stress (CS) significantly increased the incidence, number and severity of gastric ulcers. All the four test compounds had, albeit in different degrees, dose-related anti-ulcerogenic effect. The extent of the anti-ulcerogenic effect was in the order: DCPs>DBPs>DHA≈EPA as follows in Table-7.

TABLE 7
Effects of shilajit constituents on chronic stress (CS) induced gastric
ulceration in albino rats.

| Treatment groups | | Ulcer | | |
|---|---|---|---|---|
| (mg/kg, p.o.) | n | incidence % | No. of ulcers | Severity of ulcers |
| Chronic stress (CS) | 12 | 100 | 19.8 ± 3.0 | 32.4 ± 5.1 |
| EPA₍₅₎ + CS | 10 | 70 | 16.5 ± 3.4 | 28.3 ± 7.7 |
| EPA₍₁₀₎ + CS | 10 | 60 | 14.3 ± 4.4 | 26.4 ± 6.2 |
| DHA₍₅₎ + CS | 10 | 70 | 15.8 ± 4.0 | 28.1 ± 5.9 |
| DHA₍₁₀₎ + CS | 10 | 60 | 14.7 ± 3.8 | 25.0 ± 5.2 |
| DBPS₍₅₎ + CS | 10 | 50^{a} | 11.7 ± ^{b}3.1^{a} | 13.2 ± 3.0^{b} |
| DBPs₍₁₀₎ + CS | 10 | 40^{a} | 8.2 ± 2.2^{b} | 9.7 ± 2.0^{b} |
| DCPs₍₅₎ + CS | 10 | 30^{a} | 9.0 ± 2.8^{b} | 12.1 ± 2.3^{b} |
| DCPs₍₁₀₎ + CS | 10 | 20^{a} | 7.3 ± 1.8^{b} | 8.1 ± 2.0^{b} |

^{a}p < 0.05 vs CS group (chi square test);
^{b}p < 0.05 vs CS group.

Chronic stress (CS) caused marked depletion of adrenal gland ascorbic acid and corticosterone concentrations with concomitant increase in plasma corticosterone levels. These findings also suggest that the stress protocol used in this study induced pronounced stress. As expected, all the four test compounds (EPA, DHA, DBPs, DCPS) reversed, to different extents, these stress- induced adverse effects in a dose related manner; their stress- attenuating actions, in doses used, had no per se effect on the indices of stress investigated as follows in Table 8.

TABLE 8
Effects of Shilajit constituents on chronic stress (CS) induced alteration
of adrenal gland ascorbic acid and corticosterone concentrations and
plasma corticosterone level

| | | | Adrenal | Plasma |
|---|---|---|---|---|
| Groups | | Adrenal ascorbic | corticosterone | corticosterone |
| (mg/kg, p.o.) | n | acid (μg/100 mg) | (μg/100 mg) | (μg/dL) |
| Vehicle | 8 | 300.2 ± 38.4 | 4.4 ± 0.7 | 14.0 ± 1.3 |
| EPA₍₅₎ | 6 | 308.8 ± 28.7 | 5.7 ± 1.4 | 15.0 ± 0.6 |
| EPA₍₁₀₎ | 6 | 310.5 ± 26.0 | 5.2 ± 0.8 | 15.5 ± 1.1 |
| DHA₍₅₎ | 6 | 309.4 ± 30.4 | 4.8 ± 1.2 | 15.0 ± 0.9 |
| DHA₍₁₀₎ | 6 | 308.9 ± 27.4 | 5.5 ± 1.0 | 14.7 ± 1.0 |
| DBPs₍₅₎ | 6 | 309.1 ± 25.8 | 5.0 ± 1.3 | 15.7 ± 1.4 |
| DBPs₍₁₀₎ | 6 | 315.5 ± 25.5 | 5.4 ± 1.7 | 14.9 ± 1.5 |
| DCPs₍₅₎ | 6 | 308.5 ± 25.5 | 4.9 ± 0.8 | 14.7 ± 1.0 |
| DCPs₍₁₀₎ | 6 | 312.5 ± 26.0 | 5.1 ± 1.5 | 15.3 ± 1.5 |
| Chronic | 12 | 114.7 ± 16.0^{a} | 1.7 ± 0.5^{a} | 28.0 ± 3.0^{a} |
| stress (CS) | | | | |
| EPA₍₅₎ + CS | 6 | 138.5 ± 18.2 | 2.3 ± 0.8 | 22.1 ± 2.9 |
| EPA₍₁₀₎ + CS | 6 | 144.2 ± 14.7^{b} | 2.9 ± 0.7^{b} | 18.3 ± 1.8^{b} |
| DHA₍₅₎ + CS | 6 | 140.7 ± 20.5 | 2.5 ± 1.0 | 22.5 ± 3.5 |
| DHA₍₁₀₎ + CS | 6 | 148.0 ± 16.7^{b} | 2.8 ± 1.0^{b} | 17.9 ± 0.9^{b} |
| DBPs₍₅₎ + CS | 6 | 173.4 ± 18.2^{b} | 3.0 ± 1.4^{b} | 17.3 ± 0.7^{b} |
| DBPs₍₁₀₎ + CS | 6 | 198.5 ± 20.7^{b} | 3.2 ± 1.1^{b} | 16.8 ± 1.0^{b} |
| DCPs₍₅₎ + CS | 6 | 200.3 ± 25.2^{b} | 3.5 ± 1.0^{b} | 14.7 ± 1.1^{b} |
| DCPs₍₁₀₎ + CS | 6 | 242.2 ± 27.3^{b} | 3.9 ± 0.8^{b} | 14.0 ± 1.8^{b} |

^{a}p < 0.05 vs vehicle-control group;
^{b}p < 0.05 vs CS group

The effects of DBPs and DCPs on chronic stress induced suppression of humoral immunity in rats (Table-9) and in rat brain frontal cortex SOD, CAT, GPx and LPO activities (Table-10) established the major bioactivity-contribution of DCPs to shilajit.

TABLE 9
Effects of DBPs (1:1 mixture of 3-OH and 3,8-(OH)₂ dibenzo-alpha-
pyrones) and DCPs on CS - induced perturbations in rat brain frontal
cortex SOD, CAT, GPx and LPO activities^{a}.

| Treatment | | | | LPO (n mol |
|---|---|---|---|---|
| groups | SOD (μg/mg | CAT (μg/mg | GPX (μg/mg | TBARS/gm |
| (mg/Kg, p.o) | protein) | protein) | protein) | tissue) |
| Vehicle | 16.8 ± 1.4 | 20.2 ± 1.9 | 0.08 ± 0.02 | 3.32 ± 0.6 |
| Chronic | 30.9 ± 1.6^{b} | 9.6 ± 0.8^{b} | 0.02 ± 0.01^{b} | 7.4 ± 0.9^{b} |
| stress (CS) | | | | |
| DBPs | 22.0 ± 0.9^{c} | 12.8 ± 0.6^{c} | 0.03 ± 0.009 | 5.46 ± 0.7^{c} |
| (5) + CS | | | | |
| DBPs | 20.4 ± 0.8^{c} | 14.6 ± 0.8^{c} | 0.05 ± 0.008^{c} | 4.32 ± 0.8^{c} |
| (10) + CS | | | | |
| DCPs | 19.4 ± 0.9^{c} | 15.4 ± 1.2^{c} | 0.05 ± 0.06^{c} | 4.42 ± 0.09^{c} |
| (1.0) + CS | | | | |
| DCPs | 17.4 ± 1.1^{c} | 17.8 ± 0.9^{c} | 0.07 ± 0.1^{c} | 1.22 ± 0.08^{c} |
| (2.0) + CS | | | | |

^{a}= Data are means ± SEM; n = 8 to 10 replicates.
^{b}= p < 0.05 vs vehicle control group.
^{c}= p < 0.05 vs chronic stress group (CS).
SOD, superoxide dismutase,
CAT, catalase.
GPx, glutathione peroxidase.
LPO, lipid peroxidation

Test drugs were administered 14 days concomitant with stress procedure.

TABLE 10
Effects of DBPS and DCPs on chronic stress-induced suppression of
humoral immunity in rats^{a}.

| Treatment | Detectable level of | | |
|---|---|---|---|
| groups | haemagglutination titre to SRBC | | |
| (mg/Kg, p.o.) | ½- 1/16 | 1/32- 1/128 | 1/256- 1/512 |
| Vehicle | — | 74 | 26 |
| Chronic stress | 62^{b} | 38^{b} | — |
| DBPs(5) + CS | 48 | 52 | — |
| DBPs (10) + CS | 32 | 68^{c} | — |
| DCPs (1.0) + CS | 26 | 62^{c} | — |
| DCPs (2.0) + CS | 22^{c} | 72^{c} | — |

^{a}result are expressed in %; n = 8 to 10 replicate;
^{b}= p < 0.05 vs vehicle-treated control group.
^{c}= p < 0.05 vs chronic stress group (CS).
(Chi-square test). Animals were bled on day 14 after sensitization with SRBC on day 1.

### EXAMPLE 10

### Effect of DCPs on Arachidonic Acid Metabolism

The anti-inflammatory effects of shilajit and its major bioactive constituents, DCPs, were evaluated by using arachidonic acid (AA) metabolism. The effect of shilajit on AA metabolism was tested in isolated human neutrophils. Shilajit and DCPs both inhibited the biosynthesis of AA-lipoxygenase pathway products, namely, leukotriene-B₄ (LTB₄), 5-hydroxyeicosatetraenoic acid (5-HETE), 12- hydroxyeicosatetraenoic acid (12-HETE) and also inhibited the biosynthesis of the cycloxygenase product, 12-hydroxyheptadecatrienoic acid (12-HHT), in a dose dependant manner. Maximum inhibitory effects were observed at a concentration of 50 μg/ml of shilajit, while in case of DCPs, it was only 10 μg/ml. A 1:4 combination of 3,8-dihydroxydibenzo-alpha-pyrone (DBP) and fusoms exhibited similar equi-active effect at a concentration of 20 μg/ml. Fusoms of shilajit are used as an efficient systemic drug delivery agent (Ghosal, 2003). These findings suggest that the inhibition of synthesis of leukotrienes (and equivalents) by shilajit and its major bioactives (DCPs) is responsible for their therapeutic action, e.g., in the treatment of bronchial asthma.

The results as shown in Tables 7 and 8 suggest that DBPs (1:1 mixture of 3-hydroxy- and 3,8 -dihydroxydibenzo-alpha-pyrone) are biologically more active than either of its precursors, namely, EPA (eicosapentaenoic acid) or DHA (docosahexaenoic acid) while DCPs are the most active among the bioactive agents of shilajit. Similar graded effects of DBPs and DCPs were observed on chronic stress (CS)-induced perturbations in rat brain antioxidant enzymes and LPO activities (Table-9) and CS-induced suppression of humoral immunity in rats (Table-10). The Significance of DCPs in living system and the fate of DCPs after oral administration to experimental animals are also shown.

### EXAMPLE 11

### Antioxidative Actions of DCPs

Inhibition of Fe-ADP-Ascorbate Induced Lipid Peroxidation (LPO) in Rat Brain by DBP and DCPs

Albino rats (Sprague Dawley strain) were sacrificed by cervical dislocation and decapitation. Brains were dissected out and 10% w/v homogenate was prepared in 0.15 M KCl. The brain homogenate was centrifuged at 1500 rpm for 10 minutes and the supernatant was used for the study. The incubation mixture contained in a final volume of 1 ml., brain homogenate (500 μl), distilled water (100 μl) or test compounds dissolved in solvents at different concentrations (10 to 100 μg/ml of the final volume). Peroxidation was initiated by adding FeCl₃ (100 μM), ADP (1 mM) and ascorbate (100 μM) to give the final concentration stated. After incubating at 37° C. for 30 minutes, the reaction was stopped by adding acetic acid buffer (1.5 ml, pH 3.5) and thiobarbituric acid solution (1.5 ml, pH 7.4) to 1 ml of LPO mixture. The reaction mixture was heated at 85° C. for 30 minutes, cooled, centrifuged (2000 rpm for 10 minutes) and the absorbance of the supernatant was measured at 532 nm. IC₅₀ values were calculated in the usual way by plotting the concentration of the test compounds versus percent inhibition of LPO.

### EXAMPLE 12

### Metal-Ion Chelating and Scavenging Actions of DCPs

This was determined by the stability of metal-ion complexes/conjugates of DCPs and their capacity to scavenging/chelating loose metal ions.

The ion-exchange equilibrium method of Schnitzer and Skinner (1966) was used for the abovementioned determination. Briefly, amounts of DCPs ranging from 10 to 50 mg were weighed into 50 ml volumetric flasks and dissolved in approximately 40 ml of distilled water. To each flask, 5 ml of 1N-KCI solution was added. One-gram quantities of K-saturated Dowex-50 resin (20-50 mesh, Bio-RAD Laboratories) were weighed into 125 ml of ground glass-stoppered Erlenmeyer flasks. The solution containing the natural DCP-metal ion conjugates (Fe, Cu, Zn), admixed with KCl, were transferred to these flasks and shaken at 24±1° C. for 1 hour. In a separate experiment, known amounts (approx. 500 μg) of aqueous solutions of MnCl₂, MoCl₃ and WCl₄ were added separately, to aqueous solutions of the DCP-KCl. The mixtures were shaken as before and the stability constants were determined as follows. The exchange resin was then removed by filtration. The filtrates and washings, containing metal ions, Fe²⁺, Cu²⁺, Zn²⁺, and those of the added metal ions, were analyzed by Atomic Absorption Spectroscopy (Techtron AA-3 Atomic Absorption Spectrophotometer).

At pH 3.5, log stability constants for the different DCP-metal ion complexes were: DCP-Cu, 3.44; DCP-Fe, 2.83; DCP-Zn, 1.47. The order of stability of the different metal ions was (expressed in the decreasing order): Cu²⁺>Fe²⁺>Mn³⁺>Zn²⁺>Mo³⁺>W⁴⁺.

The results as shown in Tables 7 and 8 suggest that DBPs (1:1 mixture of 3-hydroxy- and 3,8 -dihydroxydibenzo-alpha-pyrone) are biologically more active than either of its precursors, namely, EPA (eicosapentaenoic acid) or DHA (docosahexaenoic acid) while DCPs are the most active among the bioactive agents of shilajit. Similar graded effects of DBPs and DCPs were observed on chronic stress (CS)-induced perturbations in rat brain antioxidant enzymes and LPO activities (Table-9) and CS-induced suppression of humoral immunity in rats (Table-10). Stress begets oxidative stress. The antioxidant actions of DCPs are pronounced as shown in Tables 11 and 12 which shows the significance of DCPs in living systems and the fate of DCPs after oral administration in experimental animals.

TABLE 11
Effect of DBP and DCPs on LPO

| Test Compound | IC₅₀ (μg/ml) |
|---|---|
| 3,8-Dihydroxydibenzo-alpha-pyrone | 30 |
| DCPs* (from shilajit) | 4 |
| Vitamin E acetate | 56 |
| Ascorbic acid | 70 |

*AcMe pptd. Note the significant antioxidant effect of DCPs

TABLE 12
Antioxidant activity of DCPs.

| ROS Captodative Activity | | RNS Captodative Activity | |
|---|---|---|---|
| (IC₅₀) | | (EC₅₀) | |
| | In terms of protein | | In terms of protein |
| | content of DCPs | | content of DCPs |
| | (60% protein in | | (60% protein in |
| Total DCPs | DCPs) | Total DCPs | DCPs) |
| 15 mcg/ml | 9 μg/ml | 80.80 μg/ml | 48.48 μg/ml |

Note:
In terms of μM of amounts, the antioxidant activities of DCPs are highly significant.

### EXAMPLE 13

### Personal Care/Cosmetic Formulations

A. SKIN REJUVENATING (O/W) LOTION

| Ingredients | % w/w |
|---|---|
| Phase A | |
| Polyglyceryl-3 Methyl Glucose Distearate | 3.50 |
| Glyceryl Stearate, PEG-100 Stearate | 2.50 |
| Dicapryl ether | 5.00 |
| Coco-Caprylate/Caprate | 5.00 |
| Propylene Glycol Dicaprylate/Dicaprate | 3.00 |
| Almond Oil | 2.00 |
| Cetyl alcohol | 1.50 |
| DCPs (present invention) | 2.00 |
| Phase B | |
| Glycerin | 3.00 |
| Propylene glycol | 3.00 |
| Allantoin | 0.20 |
| Methylparaben | 0.15 |
| Water, deionized | q.s. |
| Phase C | |
| Phenoxyethanol and Isopropylparaben and | 0.50 |
| Isobutylparaben and Butylparaben | |
| Total | 100.00 |

Procedure

Combine A, stir and heat to 65° C. Combine B, stir and heat to 65° C. Add A to B while stirring. Homogenize at moderate speeds to avoid foaming, while allowing mixture temperature to cool to 40° C. Add C, homogenize. Stir gently until mixture is homogenous.

B. SUNSCREEN O/W LOTION (SPF 15)

| Ingredients | % w/w |
|---|---|
| Phase A | |
| Propylene Glycol Isoceteth-3 Acetate | 5.00 |
| Octyl methoxycinnamate | 7.50 |
| Benzophenone-3 | 3.00 |
| Homomenthyl Salicylate | 7.00 |
| Steareth-2 | 0.40 |
| Steareth-10 | 0.80 |
| Acrylates/C. sub. 10-30 Alkyl Acrylate | 0.18 |
| Crosspolymer | |
| Synthetic Wax | 0.80 |
| Dimethicone | 1.00 |
| DCPs (present invention) | 1.00 |
| Phase B | |
| Demineralized water | q.s. |
| Phase C | |
| Demineralized water | 19.82 |
| Phenylbenzimdazole sulfonic acid | 1.00 |
| Propylene glycol | 2.00 |
| Triethanolamine | 0.90 |
| Propylene Glycol and DMDM Hydantoin | 1.00 |
| and Methylparaben | |
| Total | 100.00 |

Procedure

Combine A, stir and heat to 80° C. Heat B to 80° C. Add A to B while stirring with a propeller mixer. Continue stirring A/B for 20 minutes while maintaining the temperature between 70-75° C. Combine C, heat and stir to 45° C. until dissolved. Add C to A/B with agitation. Qs water. Gently homogenize A/B/C allowing mixture to cool to room temperature. Adjust pH to ˜6.5, if necessary, with TEA. Use high shear spray device to dispense.

C. LIQUID FOUNDATION
A liquid foundation having the following formulation
was prepared according to the following method.

| S. No. | Ingredients | % w/w |
|---|---|---|
| 1 | Lanolin | 7.00 |
| 2 | Liquid Paraffin | 5.00 |
| 3 | Stearic Acid | 2.00 |
| 4 | Cetanol | 1.00 |
| 5 | Glycerin | 5.00 |
| 6 | Triethanolamine | 1.00 |
| 7 | Carboxy Methyl Cellulose | 0.70 |
| 8 | Deminaralized Water | q.s. |
| 9 | Mica | 15.00 |
| 10 | Talc | 6.00 |
| 11 | Titanium Oxide | 3.00 |
| 12 | Coloring Pigment | 6.00 |
| 13 | DCPs (present invention) | 0.50 |
| 14 | Ultraviolet Screening Agent | q.s. |
| 15 | Perfume | q.s. |

Procedure
A. The components (1) to (4) were mixed and dissolved together.
B. The components (9) to (12) were added to and uniformly admixed with the foregoing mixture A.
C. The components (5) to (8) were uniformly dissolved together and the resulting mixture was maintained at 70° C.
D. The foregoing mixture C was added to and uniformly admixed with the foregoing mixture B to give an emulsion.
E. After cooling the foregoing mixture D, the components (13) to (15) were added thereto to give a liquid foundation.

It was found that the liquid foundation prepared in Example 3 has excellent stability over time. Application of this foundation to the skin could prevent the occurrence of any sun-induced wrinkle.

D. MOISTURE RECOVERY BODY LOTION

| Ingredients | % w/w |
|---|---|
| Phase A | |
| Demineralized Water | 76.45 |
| PVM/MA Decadiene Crosspolymer | 0.25 |
| Disodium EDTA | 0.15 |
| Hexylene Glycol | 2.00 |
| Allantoin | 0.10 |
| Phase B | |
| Glyceryl Stearate (and) Behenyl Alcohol (and) | 3.00 |
| Palmitic Acid (and) Stearic Acid (and) Lecithin | |
| (and) Lauryl Alcohol (and) Myristyl Alcohol | |
| (and) Cetyl Alcohol | |
| Isopropyl myristate | 3.00 |
| Octylhydroxy Stearate | 5.00 |
| Isostearyl Neopentanoate | 4.00 |
| Phase C | |
| Sodium Hydroxide (10% Aq. Soln.) | 0.40 |
| Phase D | |
| Glycerin (and) Glyceryl Polyacrylate | 2.00 |
| Phenyl Trimethicone | 1.00 |
| Cyclopentasiloxane | 1.00 |
| DCPs (present invention) | 0.50 |
| Phase E | |
| Propylene Glycol (and) Diazolidinyl Urea (and) | 0.50 |
| Iodopropynyl Butylcarbamate | |
| Phenoxyethanol (and) Isopropylparaben (and) | 0.50 |
| Isobutylparaben (and) Butylparaben | |
| Fragrance | 0.15 |
| Total | 100.00 |

Procedure:

1. Combine ingredients in Phase A and heat to 80° C. for 45 minutes with mixing.

2. Combine ingredients in Phase B. Heat and mix to 75° C.-80° C.

3. Add Phase B to Phase A under homogenization. Homogenize until uniform.

4. Add Phase C to Phases A&B. Take off homomix, start cooling. Switch to propeller mixing to 40° C.

5. Add Phase D ingredients at 40° C., one by one and mix well between each addition.

6. Add Phase E at 35° C. QS for water loss.

E. HAIR SHINE OIL

| Ingredients | % w/w |
|---|---|
| Phase A | |
| DCPs (present invention) | 0.25 |
| Lauryl Lactate | 3.00 |
| Phase B | |
| SD Alcohol 40-B (200 proof) | 16.75 |
| C12-15 Alkyl Benzoate | 10.00 |
| Cyclopentasiloxane | 59.00 |
| Phenyl Trimethicone | 10.00 |
| Phenoxyethanol (and) | 1.00 |
| Isopropylparaben (and) | |
| Isobutylparaben (and) Butylparaben | |
| Total | 100.00 |

Procedure:

1. Add DCPs to Lauryl lactate in a small mixing vessel. Heat the mixture to 60-70° C. Mix well with slow agitation until homogenous. Cool down to 30-35° C. while agitating.

2. Add Alcohol into a separate, larger vessel.

3. When Phase A is at 30-35° C. add Phase A to the alcohol. Mix well until homogenous. Add remaining ingredients in order with thorough mixing between each until homogenous.

4. Add the preservative. Mix well until homogenous.

F. SKIN BRIGHTENING/LIGHTENING LOTION FOR FACE

| Ingredients | % w/w |
|---|---|
| Phase A | |
| Water (demineralized) | 65.97 |
| Disodium EDTA | 0.10 |
| Propylene Glycol | 2.00 |
| Sorbitol | 2.00 |
| Sodium Lauryl Sulfate | 0.15 |
| Phase B | |
| Glyceryl stearate | 5.00 |
| Stearic acid | 1.00 |
| Avocado oil | 10.00 |
| Almond oil | 5.00 |
| Beeswax | 1.50 |
| Phase C | |
| Water (demineralized) | 5.00 |
| DCPs (present invention) | 1.00 |
| Phase D | |
| Triethanolamine | 0.28 |
| Phase E | |
| Propylene glycol, DMDM | 1.00 |
| Hydantoin, Methylparaben | |
| Total | 100.00 |

Procedure

Combine A and heat to 70-75° C. Combine B and heat to 70-750° C. Add B to A while stirring. Add phase C at 30° C. Adjust pH to 5.0-6.0 with phase D. Add phase E. Mix until uniform.

### EXAMPLE 14

### Pharmaceutical/Nutritional Formulations

A. TABLETS AND CAPSULES OF THE INVENTION

| Ingredient | Quantity per Tablet/Capsule | |
|---|---|---|
| 1. DCPs | 0.10-50.00% | by weight |
| 2. Avicel pH 101 | 200.00 | mg |
| 3. Starch 1500 | 189.00 | mg |
| 4. Stearic acid, N. F. (powder) | 8.60 | mg |
| 5. Cab-O-Sil | 2.00 | mg |

Note:
The target weight of tablet/capsule is 400 mg; Avicel pH 101 and Starch may be adjusted suitably to reach the target weight. The blended material can be filled into appropriate capsules.

B. ANTI-STRESS SUPPORT TABLETS/CAPSULES
OF THE INVENTION

| Ingredient | Quantity per Tablet/Capsule |
|---|---|
| 1. DCPs | 0.10-50.00% by weight |
| 2. Cellulose | q. s. |
| 3. Magnesium stearate | q. s. |
| 4. Gelatin | q. s. |

C. CARDIO-VASCULAR SUPPORT TABLETS
OF THE INVENTION

| | Quantity per Tablet/Capsule | |
|---|---|---|
| Ingredient | | |
| 1. DCPs | 10.0-50.00% | by weight |
| 2. Vitamin A (Beta Carotene) | 45,000 | IU |
| 3. Vitamin B-1 (Thiamin) | 25 | mg |
| 4. Inositol Hexanicotinate | 50 | mg |
| 5. Vitamin B-6 (Pyridoxine HCL) | 25 | mg |
| 6. Vitamin B-12 (Cyanocobalamin) | 500 | mcg |
| 7. Folic Acid | 800 | mcg |
| 8. Vitamin C (Magnesium Ascorbate) | 150 | mg |
| 9. Vitamin E D-alpha Tocophery (Natural) | 400 | IU |
| 10. Copper (Sebacate) | 750 | mcg |
| 11. Magnesium (Ascorbate, Taurinate, | 30 | mg |
| and Oxide) | | |
| 12. Potassium (Citrate) | 10 | mg |
| 13. Selenium (L-Selenomethionine) | 200 | mcg |
| 14. Silica (from 400 mg of Horsetail Extract) | 10 | mg |
| Other Ingredients and Herbs: | | |
| 15. Coenzyme Q10 (Ubiquinone) | 10 | mg |
| 16. L-Carnitine L-Tartrate | 50 | mg |
| 17. Hawathorn Berry Extract | 40 | mg |
| 19. Grape Seed Extract | 10 | mg |
| 20. L-Proline | 50 | mg |
| 21. L-Lysine (HCL) | 50 | mg |
| 22. N-Acetyl Glucosamine | 50 | mg |
| 23. Bromelain (2,000 GDU per g) | 120 | mg |
| 24. Taurine (Magnesium Taurinate) | 50 | mg |
| 25. Inositol (Hexanicotinate) | 10 | mg |

D. MULTI-VITAMIN & MINERAL SUPPLEMENT TABLETS OF THE
INVENTION

| | | Quantity per | |
|---|---|---|---|
| Ingredient | | Tablet | |
| 1. | DCPs | 0.50-30.00% | |
| | | by weight | |
| 2. | Vitamin A (beta carotene) | 25,000 | IU |
| 3. | Vitamin A (palmitate) | 10,000 | IU |
| 4. | Vitamin B-1 (Thiamin Nitrate) | 10 | mg |
| 5. | Vitamin B-2 (Riboflavin) | 10 | mg |
| 6. | Inositol Hexanicotinate, Niacinamide & Niacin | 20 | mg |
| 7. | Vitamin B-5 (Calcium D-Pantothenate) | 10 | mg |
| 8. | Vitamin B-6 ((Phyridoxine HCL) | 10 | mg |
| 9. | Vitamin B-12 (Cyanocobalamin) | 200 | mcg |
| 10. | Biotin | 500 | mcg |
| 11. | Folic Acid | 800 | mcg |
| 12. | Vitamin C | 180 | mg |
| | (Magnesium, Manganese & Zinc Ascorbates) | | |
| 13. | Fat-Soluble Vitamin C | 20 | mg |
| | (from 476 mg of Ascorbyl Palmitate) | | |
| 14. | Vitamin D-3 (Cholecalciferol) | 400 | IU |
| 15. | Vitamin E D-alpha Tocopheryl (Natural) | 600 | IU |
| 16. | Boron (Amino Acid Chelate) | 2 | mg |
| 17. | Calcium (Succinate, Carbonate, Malate) | 20 | mg |
| 18. | Copper (Sebacate) | 1 | mg |
| 19. | Iodine (from Kelp) 150 mcg, | 150 | mcg |
| | Magnesium (Ascorbate, Oxide, Succinate) | | |
| 20. | Manganese (Ascorbate) | 30 | mg |
| 21. | Molybdenum (Amino Acid Chelate) | 300 | mcg |
| 22. | Potassium (Succinate, alpha-Ketoglutarate) | 10 | mg |
| 23. | Selenium | 250 | mcg |
| | (L-Selenomethionine & Sodium Selenite) | | |
| 24. | Zinc (Zinc Monomethionine & Ascorbate) | 10 | mg |

Other Ingredients and Plant antioxidants: N-Acetyl Cysteine, Succinic Acid (Free Form), Choline (Bitartrate), Inositol (Hexanicotinate and Inositol), N-Acetyl Glucosamine, DMAE (Bitartrate), N-Acetyl L-Tyrosine, Coenzyme Q10, Alpha-Lipoic Acid, Quercetin, Milk Thisle Seed Extract, Grape Seed Extract, Ginkgo Biloba, Bilberry Extract.

E. ANTI-DIABETIC SUPPORT TABLETS/CAPSULES OF
THE INVENTION

| | Quantity per |
|---|---|
| Ingredient | Tablet/Capsule |
| 1. DCPs | 0.10-50.00% by weight |
| 2. Vitamin B-6 (as Pyridoxine HCI) | 10 mg |
| 3. L-Arginine | 50 mg |
| 4. L-Lysine Monohydrochloride | 50 mg |
| 5. Cellulose | q.s. |
| 6. Magnesium stearate | q.s. |
| 7. Gelatin | q.s. |

F. WEIGHT LOSS SUPPORT TABLETS OF THE INVENTION

| | Quantity per |
|---|---|
| Ingredient | Tablet/Capsule |
| 1. DCPs | 0.10-50.00% by weight |
| 2. Garcinia Cambogia Extract | 60 mg |
| 3. Bitter Orange Peel Standardized Extract | 20 mg |
| 4. Green Tea | 10 mg |
| 5. Cayenne | 15 mg |
| 6. Mustard Seed | 10 mg |
| 7. Ginger Root | 10 mg |
| 8. Piper nigrum | 10 mg |
| 9. Acetyl L-Carnitine | 10 mg |
| 10. Niacinamide | 10 mg |
| 11. Vitamin B-6 (Pyridoxine HCL) | 10 mg |

G. CHEWABLE TABLETS OF THE INVENTION

| | | Composition |
|---|---|---|
| Ingredient No. | Ingredient | (% w/w) |
| 1 | DCPs | 0.10-50.00 |
| 2 | Sodium ascorbate, USP | 12-35 |
| 3 | Avicel pH 101 | 5-15 |
| 4 | Sodium saccharin, N. F. | 0.56 |
| | (powder) | |
| 5 | DiPac | 10-30 |
| 6 | Stearic acid, N. F | 2.50 |
| 7 | Imitation orange flavor | 1.00 |
| 8 | FD&C Yellow#6 dye | 0.50 |
| 9 | Cab-O-Sil | 0.50 |

Procedure: Blend all the ingredients, except 6, for 20 min. in a blender. Screen in 6 and blend for an additional 5 min. Compress into tablets using 7/16-in standard concave tooling.

H. SYRUP OF THE INVENTION

| Ingredient No. | Ingredient | Quantity per 100 mL |
|---|---|---|
| 1 | DCPs | 0.10-50.00% by volume |
| 2 | Excipients | q.s |

I. ORAL LIQUID OF THE INVENTION

| Ingredient | | Quantity per 100 ml |
|---|---|---|
| 1. | DCPs | 0.10-50.00% by volume |
| 2. | Purified Water | q.s. |
| 3. | Excipients: Preservatives, stabilizers, | q.s. |
| | sweetners, flavors, colors, etc. | |

J. SNACK BAR WITH THE INVENTION

| In- | | |
|---|---|---|
| gre- | | |
| dient | | Quantity |
| No. | Ingredient | per 1 Kg |
| 1 | DCPs | 0.50-30.00% |
| | | by weight |
| 2 | Nutrition Blend: Calcium (Tricalcium Phosphate and | q.s |
| | Calcium Carbonate), Magnesium (Magnesium | |
| | Oxide), Vitamin A, Vitamin C, Vitamin D-3, | |
| | Vitamin B-1 (Thiamin), Vitamin B-2 (Riboflavin), | |
| | Vitamin B-6 (Pyridoxine), Vitamin B-12 | |
| | (Cyanocobalamin), Natural Vitamin (Acetate), | |
| | Niacin, Biotin, Pantothenic Acid, Zinc, Folic Acid, | |
| | Vitamin K, Selenium. Other Ingredients: Protein | |
| | Blend (Soy protein isolate, Hydrolyzed collagen, | |
| | Whey protein isolate, Calcium/Sodium Caseinate), | |
| | Glycerine, Polydextrose (fiber), Water, Cocoa | |
| | Butter, Natural Coconut Oil (non-hydronated), | |
| | Coconut, Cellulose, Cocoa Powder, Olive Oil, | |
| | Lecithin, Natural and Artificial Flavor, | |
| | Maltodextrin, Guar Gum, Citric Acid (Flavor | |
| | Enhancer), Sucralose | |

K. CEREAL WITH THE INVENTION

| Ingredient | | Quantity |
|---|---|---|
| No. | Ingredient | per 1 Kg |
| 1 | DCPs | 0.50-30.00% |
| | | by weight |
| 2 | Excipients: Whole Grain Oats, Oat Bran, | q.s |
| | Sugar, Modified Corn Starch, Brown Sugar | |
| | Syrup, Salt, Calcium Carbonate, | |
| | Trisodium Phosphate, Wheat Flour, Vitamin | |
| | E (Mixed tocopherols), Zinc & Iron (Mineral | |
| | nutrients), Niacinamide (A B Vitamins), | |
| | Vitamin B6 (Pyridoxine Hcl), Vitamin B2 | |
| | (Riboflavin), Vitamin B1 (Thiamin | |
| | Mononitrate), Vitamin A (Palmitate), Vitamin | |
| | A B (Folic acid), Vitamin B12, Vitamin D | |

L. BEVERAGE WITH THE INVENTION

| Ingre- | | |
|---|---|---|
| dient | | Quantity per |
| No. | Ingredient | 500 mL |
| 1 | DCPs | 0.50-30.00% |
| | | by volume |
| 2 | Excipients: Filtered Water, Food Starch- | q.s |
| | Modified, Citric Acid, Bitter Orange, Green Tea | |
| | Extract, Maltodextrin, Whey Protein Isolate, | |
| | High Fructose Corn Syrup and/or Sucrose and/or | |
| | Sugar, Sodium Benzoate, Caffeine, Niacin, | |
| | Glycerol Ester of Wood resin, Flavors, Colors | |

### EXAMPLE 15

### Veterinary Formulations

A. CHEWABLE TABLETS OF THE INVENTION

| Ingredient No. | Ingredient | Composition | |
|---|---|---|---|
| 1 | DCPs | 0.10-50.00% w/w | |
| 2 | Calcium (from calcium phosphate) | 600 | mg |
| 3 | Phosphorus (from calcium phosphate) | 470 | mg |
| 4 | Vitamin C | 10 | mg |
| 5 | Vitamin A | 750 | I. U. |
| 6 | Vitamin D3 | 400 | I. U. |

| | | |
|---|---|---|
| 7 | Excipients | q.s. |

Note:
Administer free choice just prior to feeding, or crumble and mix with food

B. VITAMIN TABLETS OF THE INVENTION
(PEANUT BUTTER FLAVOR)

| Ingredient | | Quantity per Tablet |
|---|---|---|
| 1. | DCPs | 0.10-50.00% by weight |
| 2. | Other Ingredients: | q.s. |
| | Brewer's Yeast Powder, Garlic, Whey, | |
| | Beef Liver, Peanut Butter, Silica Gel, | |
| | Niacin, Riboflavin, Thiamine Mononitrate, | |
| | Ascorbic acid | |

C. GRANULES OF THE INVENTION

| Ingredient | | Quantity per 4 oz. |
|---|---|---|
| 1. | DCPs | 0.10-50.00% by weight |
| 2. | Other Ingredients: | q.s. |
| | Potassium Gluconate, Wheat, Sucrose, | |
| | Hydrolyzed Vegetable Protein, | |
| | Silicone Dioxide, TBHQ (preservative) | |

D. BLOOD BUILDING POWDER OF THE INVENTION

| Ingredient | | Quantity per lb. |
|---|---|---|
| 1. | DCPs | 0.10-50.00% by weight |
| 2. | Other Ingredients: | q.s. |
| | Heme iron polypeptide, Niacin | |
| | (Vitamin B3), Vitamin E acetate, | |
| | Riboflavin (Vitamin B2), Thiamine | |
| | (Vitamin B1), Pyridoxine (Vitamin B6), | |
| | Vitamin B12, Copper Sulfate, Cobalt | |
| | sulfate, Soybean oil, Whey, Natural | |
| | sweet apple and molasses flavors | |

E. LIQUID CAPSULES OF THE INVENTION

| Ingredient | | Quantity per Capsule |
|---|---|---|
| 1. | DCPs | 0.10-50.00% by weight |
| 2. | Other Ingredients: | q.s. |
| | Safflower Oil, Gelatin, Fish Oil, Glycerin, | |
| | Borage Seed Oil, Vitamin E, Water | |

Note:
The capsules may be punctured and the liquid contents squeezed onto food, if desired.

F: ORAL LIQUID OF THE INVENTION

| Ingredient | | Quantity per 100 ml |
|---|---|---|
| 1. | DCPs | 0.10-50.00% by volume |
| 2. | Purified Water, Sugar, Sorbitol, | q.s. |
| | Polysorbate 80, Propylene glycol, | |
| | Peptones Ferric ammonium citrate, | |
| | nicotinamide, Vitamin A and D3 | |
| | concentrate, d-panthenol, Thiamine Hcl | |
| | (Vitamin B1), alpha tocopheryl acetate | |
| | (Vitamin E), saccharine sodium, | |
| | Vitamin A palmitate, Pyridoxine Hcl | |
| | (Vitamin B6), Riboflavin 5′-Phosphate | |
| | sodium (source of Vitamin B2) | |
| 3. | Excipients: Preservatives, stabilizers, | q.s. |
| | sweeteners, flavors, colors, etc. | |

G. SUSPENSION OF THE INVENTION

| Ingredient | | Quantity per | |
|---|---|---|---|
| No. | Ingredient | each oz. | |
| 1 | DCPs | 0.10-50.00% | |
| 2 | Fat (Polyunsaturated) | 45% | |
| 3 | Carbohydrate | 33% | |
| 4 | Vitamin A | 500 | I.U. |
| 5 | Vitamin D3 | 40 | I.U. |
| 6 | Vitamin E | 3 | I.U. |
| 7 | Thiamine Hcl (Vitamin B1) | 0.15 | mg |
| 8 | Riboflavin 5′Phos Na (Vitamin B2) | 0.17 | mg |
| 9 | Pyridoxine Hcl (Vitamin B6) | 0.2 | mg |
| 10 | Ascorbic acid (Vitamin C) | 6.0 | mg |
| 11 | Nicotinamide | 2.0 | mg |
| 12 | Pantothenic acid | 1.0 | mg |
| 13 | Folic acid | 0.04 | mg |

| | | |
|---|---|---|
| 14 | Sodium Benzoate | 0.1% |

H. INJECTABLE OF THE INVENTION

| Ingredient | Quantity per ml |
|---|---|
| 1. DCPs | 0.1-10% by volume |
| 2. Water for Injection, USP | q.s. |
| 3. Ingredients to maintain proper pH | q.s. |

1. A composition of dibenzo-alpha-pyrones chromoproteins (DCPs) comprising:
a. dibenzo-alpha-pyrones or their derivatives;
b. phosphocreatine;
c. chromo-peptides of molecular weights of about≦2 KD; and
d. lipids having fatty acyl esters of glycerol.

2. A composition according to claim 1 comprising said dibenzo-alpha-pyrones of formula (I) 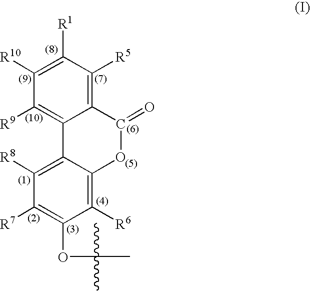
wherein:
R¹ is selected from the group consisting of H, OH, O-acyl, and O-amino-acyl; and
R¹, R ⁶, R⁷, R⁸, R⁹, and R¹⁰ are independently selected from the group consisting of H, OH, O-acyl, O-amino-acyl, and fatty acyl groups.

3. A composition according to claim 1 wherein said phosphocreatine is attached to the 3- or 8-position of said dibenzo-alpha-pyrones via an ester linkage.

4. A composition according to claim 1 wherein said chromo-peptides further comprise:
one or more amino acids;
carotenoids; and
indigoids.

5. A composition according to claim 1 wherein said chromo-proteins have a molecular weight of about 2 to about 20 KD.

6. A composition according to claim 5 wherein said chromo-proteins comprise of one or more amino acids selected from the group consisting of methionine, arginine, glycine, alanine, threonine, serine, proline, and hydroxyproline.

7. A composition according to claim 1 wherein said chromo-peptides comprise of a carotenoid moiety, said carotenoid moiety is astaxanthin and equivalents.

8. A composition according to claim 1 wherein said lipids are saturated or unsaturated fatty acids having a carbon chain length of about C₁₄ to C₂₄.

9. A composition according to claim 8 wherein said polyunsaturated fatty acid substituents have a degree of unsaturation of one to six.

10. A composition according to claim 9 wherein said polyunsaturated fatty acids are eicosapentaenoic acid and/or docosahexaenoic acid.

11. A composition according to claim 1 wherein said DCPs further comprise iron, calcium, copper, zinc, magnesium, vanadium, and/or metal ions ranging from about 1 to about 500 ppm levels.

12. A composition according to claim 1 wherein said DCPs further comprise low molecular weight ligands.

13. A skin care, hair care, pharmaceutical, or nutritional or veterinary formulation comprising the composition of claim 1 present therein in an amount of about 0.05 to about 50% by weight.

14. A skin care or protection formulation according to claim 13 where said skin care or protection formula is in the form of a lotion, cream, gel or spray, and said composition is present in an amount of about 0.05 to about 5% by weight.

15. A pharmaceutical formulation according to claim 13 wherein said pharmaceutical formulation is in the form of a tablet, syrup, elixir or capsule.

16. A nutritional formulation according to claim 13 wherein said nutritional formulation contains about 0.5 to about 30% of said composition.

17. A skin care or protection formulation according to claim 14, further comprising a cosmetically acceptable carrier and at least one cosmetic adjuvant selected from the group consisting of sunscreens, antioxidants, preservatives, self-tanning agent, perfumes, oils, waxes, propellants, waterproofing agents, emulsifiers, thickeners, humectants, and emollients.

18. A pharmaceutical formulation according to claim 15 further comprising pharmaceutically acceptable carriers.

19. A nutritional formulation according to claim 16 further comprising nutritionally acceptable carriers.

20. A process for isolating DCP compositions according to claim 1 from shilajit compositions comprising at least 0.5%-10% w/w dibenzo-alpha-pyronechromoproteins, said process comprising the steps of:
1) powdering native shilajit rock material and extracting it successively with hot ethyl acetate and methanol to remove the soluble low and medium molecular weight organic compounds by filtration;
2) triturating said ethyl acetate and methanol insoluble material with hot water and then citrate buffer of pH 5.0;
3) filtering the combined extract-mixture to remove insoluble substances comprising polymeric humic materials, minerals and metal ion salts;
4) gradually saturating the combined aqueous filtrate with increasing concentrations of ammonium sulphate to obtain purple-brown precipitate of mixture of DCPs, or concentrating said combined aqueous solution and adding acetone to precipitate DCPs as brownish-red or off-white precipitate and filtering said DCPs and evaporating the filtrate to obtain an additional lot of mixture of DCPs of lesser complexities; and
5) fractionating the purple-brown solid residues, obtained from ammonium sulphate saturation by Sephadex gel-filtration and electrophoresis to isolate DCP compositions from shilajit.

21. A process according to claim 20 wherein said shilajit composition is about 12% to about 40% w/w dibenzo-alpha-pyronechromoproteins.

22. A process for isolating DCP compositions according to claim 1 from fossils of ammonites, said process comprising the steps of:
1) powdering ammonite fossil materials and extracting it successively with hot ethyl acetate and methanol to remove the soluble low and medium molecular weight organic compounds by filtration;
2) triturating said ethyl acetate and methanol insoluble material with 0.1 N HCl;
3) filtering the aqueous acidic extract to remove insoluble substances comprising polymeric humic materials and dissolving in minimum volume of water;
4) gradually saturating the aqueous solution with increasing concentrations of ammonium sulphate to obtain purple-brown precipitate of mixture of DCPs, or concentrating said combined aqueous solution and adding acetone to precipitate DCPs as brownish-red or off-white precipitate and filtering said DCPs and evaporating filtrate to obtain an additional lot of mixture of DCPs of lesser complexities; and
5) fractionating the purple-brown solid residues, obtained from ammonium sulphate saturation by Sephadex gel-filtration and electrophoresis to isolate DCP compositions from fossils of ammonites.

23. A process for isolating DCP compositions according to claim 1 from fossils of corals, said process comprising the steps of:
1) powdering coral fossil materials and extracting it successively with hot ethyl acetate and methanol to remove the soluble low and medium molecular weight organic compounds by filtration;
2) triturating said ethyl acetate and methanol insoluble material with 0.1 N HCI;
3) filtering the aqueous acidic extract to remove insoluble substances comprising polymeric humic materials and dissolving in minimum volume of water,
4) gradually saturating the aqueous solution with increasing concentrations of ammonium sulphate to obtain purple-brown precipitate of mixture of DCPs, or concentrating said combined aqueous solution and adding acetone to precipitate DCPs as brownish-red or off-white precipitate and filtering said DCPs and evaporating filtrate to obtain an additional lot of mixture of DCPs of lesser complexities; and
5) fractionating the purple-brown solid residues, obtained from ammonium sulphate saturation by Sephadex gel-filtration and electrophoresis to isolate DCP compositions from fossils of corals.

24. A process for isolating DCP compositions according to claim 1 from invertebrates, said process comprising the steps of:
1) extracting body flesh with hot ethyl acetate to remove low molecular weight free organic compounds and lipids as the soluble fraction;
2) extracting said ethyl acetate with Bligh and Dyer solvent system;
3) evaporating said Bligh and Dyer solvent extractive under reduced pressure and dissolving in minimum volume of water;
4) gradually saturating said water with increasing concentrations of ammonium sulphate to obtain purple-brown precipitate of mixture of DCPs, or concentrating said combined water solution and adding acetone to precipitate DCPs as brownish-red or off-white precipitate and filtering said DCPs and evaporating filtrate to obtain an additional lot of mixture of DCPs of lesser complexities; and
5) fractionating the purple-brown solid residues, obtained from ammonium sulphate saturation by Sephadex gel-filtration and electrophoresis to isolate DCP compositions from invertebrates.

25. A method for treating chronic stress, comprising administering to a patient in need thereof a therapeutically effective amount of a composition according to claim 1.

26. A composition comprising of dibenzo-alpha-pyrone chromoproteins (DCPs) of formula (I): 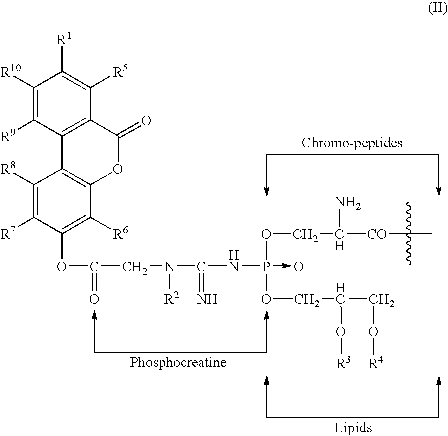
wherein:
R¹ is selected from the group consisting of H, OH, O-acyl, and O-amino-acyl;
R² is selected from H and CH₃;
R³ is selected from H and fatty acids;
R⁴ is selected from Hand fatty acids; and
R⁵, R⁶, R⁷, R⁸, R⁹, and R¹⁰ are independently selected from the group consisting of H, OH, O-acyl, O-amino-acyl, and fatty acyl group.

27. A composition according to claim 26 wherein said DCPs further comprise iron, calcium, copper, zinc, magnesium, vanadium, and/or metal ions ranging from 1 to 500 ppm levels.

28. A composition of claim 27 wherein said DCPs further comprise low molecular weight ligands.

29. A skin care, hair care, pharmaceutical, or nutritional formulation comprising the composition of claim 26 present therein in an amount of about 0.05 to 50% by weight.

30. A method for treating chronic stress disorders, comprising administering to a patient in need thereof a therapeutically effective amount of a composition according to claim 26.

31. A method for increasing a cognition effect of learning, comprising administering to a patient in need thereof a therapeutically effective amount of a composition according to claim 13.

32. A method for treating stress disorders, comprising administering to a patient in need thereof a therapeutically effective amount of a composition according to claim 13.

33. A method according to claim 32, wherein the disorder is selected from anxiety induced stress, depression induced stress, thermic change induced stress, gastric ulcer induced stress, convulsion induced stress, and adrenocortial induced stress.

34. A method for modulation of an immune system by increasing antioxidant defense enzymes selected from the group consisting of super oxide dismutase (SOD), catalase, and glutathione peroxidase comprising administering to a patient in need thereof a therapeutically effective amount of a composition according to claim 13.

35. A method for increasing a cognition effect of learning, comprising administering to a patient in need thereof a therapeutically effective amount of a composition according to claim 29.

36. A method for treating stress disorders, comprising administering to a patient in need thereof a therapeutically effective amount of a composition according to claim 29.

37. A method according to claim 36, wherein the disorder is selected from anxiety induced stress, depression induced stress, thermic change induced stress, gastric ulcer induced stress, convulsion induced stress, and adrenocortial induced stress.

38. A method for modulation of an immune system by increasing antioxidant defense enzymes selected from the group consisting of super oxide dismutase (SOD), catalase, and glutathione peroxidase comprising administering to a patient in need thereof a therapeutically effective amount of a composition according to claim 29.
